(19) United States
(12) Patent Application Publication
(10) Pub. No.: US 2025/0090617 A1
(43) Pub. Date: Mar. 20, 2025

(54) FORMULATIONS FOR TREATING OBESITY AND DIABETES AND FOR PROVIDING OTHER HEALTH RELATED BENEFITS

(71) Applicant: Healthrite Partners, LLC, Redondo Beach, CA (US)

(72) Inventors: Danny Petrasek, Los Angeles, CA (US); Ferid Murad, Menlo Park, CA (US)

(21) Appl. No.: 18/963,422

(22) Filed: Nov. 27, 2024

Related U.S. Application Data
(63) Continuation-in-part of application No. 18/145,781, filed on Dec. 22, 2022.
Application No. 18/145,781 is a division of application No. 16/984,610, filed on Aug. 4, 2020, now Pat. No. 11,541,094.
(60) Provisional application No. 62/883,270, filed on Aug. 6, 2019.

(51) Int. Cl. A61K 36/736; A23L 29/00; A23L 33/00; A23L 33/105; A23L 33/15; A61K 31/05; A61K 31/375; A61K 31/733; A61K 36/87; A61K 36/9066

(52) U.S. Cl. CPC A61K 36/736; A23L 29/035; A23L 33/105; A23L 33/15; A23L 33/30; A61K 31/05; A61K 31/375; A61K 31/733; A61K 36/87; A61K 36/9066

(57) ABSTRACT

Disclosed herein are formulations, methods, and kits of increasing GLP-1 levels. Some embodiments of the formulations are useful for inhibiting, ameliorating, delaying the onset of, reducing the likelihood of, treating, and/or preventing obesity. The formulations are also useful for inhibiting, ameliorating, delaying the onset of, reducing the likelihood of, treating, and/or preventing diabetes. The formulations have at least one extract from a tart cherry, resveratrol, and L-arginine. Also disclosed are methods of making the formulations.

### PRIORITY AND CROSS-REFERENCE TO RELATED APPLICATIONS

This application is a Continuation-In-Part application of U.S. application Ser. No. 18/145,781, filed Dec. 22, 2022, which is a divisional application of U.S. application Ser. No. 16/984,610, filed Aug. 4, 2020 (now U.S. Pat. No. 11,541,094), which claims the benefit of U.S. Provisional Application No. 62/883,270, filed Aug. 6, 2019. Any and all applications for which a foreign or a domestic priority is claimed or which is/are identified in the Application Data Sheet filed herewith and is/are hereby incorporated by reference in their entireties.

### FIELD

Though not so limited, the present disclosure is generally related to formulations, methods, and kits of increasing GLP-1 levels for treatment of obesity and diabetes.

### BACKGROUND

Metabolic syndrome is a chronic condition that increases the risk of cardiovascular disease and type 2 diabetes. Metabolic syndrome is characterized by a combination of metabolic disorders, including: (1) central obesity resulting in a waist circumference greater than 102 cm in men and 88 cm in women; (2) insulin resistance wherein cells do not respond normally to insulin, causing blood sugar levels to rise; (3) hypertension (high blood pressure) defined as a systolic pressure of at least 130 mm Hg or a diastolic pressure of at least 85 mm Hg; and (4) dyslipidemia with elevated triglycerides and low high-density lipoproteins (HDL).

### SUMMARY OF SOME EXAMPLE EMBODIMENTS

In some embodiments, a method for increasing GLP-1 levels in a subject is provided.

In some embodiments, the method for increasing GLP-1 levels comprises administering to the subject a formulation comprising an effective amount of at least one extract from a tart cherry, an effective amount of resveratrol, an effective amount of L-arginine. The effective amount of each ingredient is sufficient in combination for increasing GLP-1 levels.

In some embodiments of the method for increasing GLP-1 levels, the effective amount of the at least one extract from a tart cherry ranges from about 250 mg to about 1000 mg.

In some embodiments of the method for increasing GLP-1 levels, the effective amount of the resveratrol ranges from about 100 mg to about 400 mg.

In some embodiments of the method for increasing GLP-1 levels, the effective amount of the L-arginine ranges from about 1.5 g to about 9 g.

In some embodiments of the method for increasing GLP-1 levels, the formulation further comprises an effective amount of at least one antioxidant vitamin.

In some embodiments of the method for increasing GLP-1 levels, the least one antioxidant vitamin is Vitamin C.

In some embodiments of the method for increasing GLP-1 levels, an amount of the Vitamin C ranges from about 25 mg to about 500 mg.

In some embodiments of the method for increasing GLP-1 levels, the formulation further comprises an effective amount of at least one prebiotic.

In some embodiments of the method for increasing GLP-1 levels, the at least one prebiotic is inulin.

In some embodiments of the method for increasing GLP-1 levels, the effective amount of inulin ranges from about 45 mg to about 4500 mg.

In some embodiments, the method for increasing GLP-1 levels inhibits ameliorates, delays an onset of, reduces a likelihood of, treats, and/or prevents diabetes in the subject.

In some embodiments, the method for increasing GLP-1 levels inhibits ameliorates, delays an onset of, reduces a likelihood of, treats, and/or prevents obesity in the subject.

In some embodiments, a method for increasing GLP-1 levels in a subject is provided.

In some embodiments, the method for increasing GLP-1 levels comprises administering to the subject a formulation comprising an amount of at least one extract from a tart cherry, an amount of resveratrol, an amount of L-arginine. The amounts of the at least one extract from a tart cherry, the resveratrol, and the L-arginine are, in combination, effective in increasing GLP-1 levels.

In some embodiments of the method for increasing GLP-1 levels, the amount of the at least one extract from a tart cherry ranges from about 250 mg to about 1000 mg.

In some embodiments of the method for increasing GLP-1 levels, the amount of the resveratrol ranges from about 100 mg to about 400 mg.

In some embodiments of the method for increasing GLP-1 levels, the amount of the L-arginine ranges from about 1.5 g to about 9 g.

In some embodiments of the method for increasing GLP-1 levels, the formulation further comprises an amount of at least one antioxidant vitamin.

In some embodiments of the method for increasing GLP-1 levels, the least one antioxidant vitamin is Vitamin C.

In some embodiments of the method for increasing GLP-1 levels, an amount of the Vitamin C ranges from about 25 mg to about 500 mg.

In some embodiments of the method for increasing GLP-1 levels, the formulation further comprises an amount of at least one prebiotic.

In some embodiments of the method for increasing GLP-1 levels, the at least one prebiotic is inulin.

In some embodiments of the method for increasing GLP-1 levels, the amount of inulin ranges from about 45 mg to about 4500 mg.

In some embodiments, the method for increasing GLP-1 levels inhibits ameliorates, delays an onset of, reduces a likelihood of, treats, and/or prevents diabetes in a subject.

In some embodiments, the method for increasing GLP-1 levels inhibits ameliorates, delays an onset of, reduces a likelihood of, treats, and/or prevents obesity in a subject.

In some embodiments, a formulation is provided.

In some embodiments, the formulation comprises an effective amount of at least one extract from a tart cherry, an effective amount of resveratrol, an effective amount of L-arginine, and a pharmaceutically acceptable carrier.

In some embodiments of the formulation, the effective amount of the at least one extract from a tart cherry ranges from about 250 mg to about 1000 mg.

In some embodiments of the formulation, the effective amount of the resveratrol ranges from about 100 mg to about 400 mg.

In some embodiments of the formulation, the effective amount of the L-arginine ranges from about 1.5 g to about 9 g.

In some embodiments of the formulation, the formulation further comprises an effective amount of at least one antioxidant vitamin.

In some embodiments of the formulation, the least one antioxidant vitamin is Vitamin C.

In some embodiments of the formulation, an effective amount of the Vitamin C ranges from about 25 mg to about 500 mg.

In some embodiments of the formulation, the formulation further comprises an amount of at least one prebiotic.

In some embodiments of the formulation, the at least one prebiotic is inulin.

In some embodiments of the formulation, the effective amount of inulin ranges from about 45 mg to about 4500 mg.

In some embodiments, a formulation is provided.

In some embodiments, the formulation comprises an amount of at least one extract from a tart cherry, an amount of resveratrol, an amount of L-arginine, and a pharmaceutically acceptable carrier.

In some embodiments of the formulation, the amount of the at least one extract from a tart cherry ranges from about 250 mg to about 1000 mg.

In some embodiments of the formulation, the amount of the resveratrol ranges from about 100 mg to about 400 mg.

In some embodiments of the formulation, the amount of the L-arginine ranges from about 1.5 g to about 9 g.

In some embodiments of the formulation, the formulation further comprises an amount of at least one antioxidant vitamin.

In some embodiments of the formulation, the least one antioxidant vitamin is Vitamin C.

In some embodiments of the formulation, an amount of the Vitamin C ranges from about 25 mg to about 500 mg.

In some embodiments of the formulation, the formulation further comprises an amount of at least one prebiotic.

In some embodiments of the formulation, the at least one prebiotic is inulin.

In some embodiments of the formulation, the amount of inulin ranges from about 45 mg to about 4500 mg.

### BRIEF DESCRIPTION OF THE DRAWINGS

FIG. 1 shows generation of uric acid via the purine metabolism pathway.

FIG. 2 shows generation of uric acid via the salvage pathway.

FIG. 3 shows generation of ATP from phosphocreatine.

FIG. 4 shows a schematic of the biology of GLP-1.

### DETAILED DESCRIPTION

Metabolic Syndrome is considered to be present when a subject based on established clinical criteria. Metabolic syndrome is a set of clinical conditions that occur together and is associated with an increased risk of heart disease, stroke and type 2 diabetes. The conditions include elevated blood pressure, elevated blood glucose, excess body fat, abnormal cholesterol and/or triglyceride levels, and elevated uric acid levels.

The clinical criteria for the diagnosis of metabolic syndrome, as defined in the National Cholesterol Education Program's Adult Treatment Panel III (ATP III) report (which is hereby included by reference in its entirety), include waist circumference of more than 102 cm (40 in) in men and more than 88 cm (35 in) in women; triglyceride levels of at least 150 mg per dL (1.70 mmol per L); high-density lipoprotein cholesterol levels of less than 40 mg per dL (1.04 mmol per L) in men and less than 50 mg per dL (1.30 mmol per L) in women; blood pressure of at least 130/85 mm Hg; and fasting glucose levels of at least 110 mg per dL (6.10 mmol per L) (Table 1).

TABLE 1
Definitions of metabolic syndrome

| | NCEP ATP III (2005 | | | |
|---|---|---|---|---|
| | REVISION) | WHO (1998) | EGIR (1999) | IDF (2005) |
| Absolutely | None | Insulin resistance* | Hyperinsulinemia^{±} | Central obesity |
| required | | (IGT, IFG, T2D or | (plasma insulin | (waist |
| | | other evidence of | >75^{th} percentile) | circumference^{#}) |
| | | IR) | | ≥94 cm (M), ≥80 |
| | | | | cm (F) |
| Criteria | Any three of the five | Insulin resistance | Hyperinsulinemia, | Obesity, plus two of |
| | criteria below | or diabetes, plus | plus two of the | the four criteria |
| | | two of the five | four criteria below | below |
| | | criteria below | | |
| Obesity | Waist circumference: >40 | Waist/hip ratio: | Waist | Central obesity |
| | inches (M), >35 | >0.90 (M), >0.85 | circumference: | already required |
| | inches (F) | (F); or BMI > 30 | ≥94 cm (M), ≥80 | |
| | | kg/m² | cm (F) | |
| Hyperglycemia | Fasting glucose ≥ 100 | Insulin resistance | Insulin resistance | Fasting glucose |
| | mg/dl or Rx | already required | already required | ≥100 mg/dl |
| Dyslipidemia | TG ≥ 150 mg/dl or Rx | TG ≥ 150 mg/dl or | TG ≥ 177 mg/dl or | TG ≥ 150 mg/dl or |
| | | HDL-C: <35 mg/dl | HDL-C < 39 mg/dl | Rx |
| | | (M), <39 mg/dl (F) | | |
| Dyslipidemia | HDL cholesterol: <40 | | | HDL cholesterol: |
| (second, | m/dl (M), <50 mg/dl | | | <40 mg/dl (M), <50 |
| separate | (F); or Rx | | | mg/dl (F); or Rx |
| criteria) | | | | |
| Hypertension | >130 mmHg systolic | ≥140/90 mmHg | ≥140/90 mmHg or | >130 mmHg |
| | or >85 mmHG | | Rx | systolic or >85 |
| | diastolic or Rx | | | mmHG diastolic or |
| | | | | Rx |
| Other criteria | | Microalbuminuria¹ | | |

*IGT, impaired glucose tolerance; IFG, impaired fasting glucose; T2D, type 2 diabetes; IR, insulin resistance; other evidence includes euglycemic clamp studies.
¹Urinary album in excretion of ≥ 20 μg/min or albumin-to-creatine ration of ≥ 30 mg/g.
^{±}Reliable only n patients without T2D.
^{#}Criteria for central obesity (waist circumference) are specific for each population; values given are for European men and women.
Rx, pharmacologic treatment.

Rx, pharmacologic treatment.

The etiology of the syndrome is believed to be related to at least three general features that may be interrelated: insulin resistance, dysregulation of adipose physiology and inflammation.

The prevalence of the metabolic syndrome parallels the prevalence of Type II Diabetes (DM II) and has been increasing at an accelerated rate. The incidence of DM II was in the range of 1-2% of the population at the beginning of the 20th century and currently it is estimated to be close to 10% of the population. The metabolic syndrome classification was an eventual outcome of the Framingham study to determine cardiovascular risk factors. Motivated by the death of FDR from malignant hypertension in 1945, Harvard Medical school initiated a government sponsored program, in the town of Framingham Massachusetts, in order to establish risk factors for cardiovascular disease. The Framingham study established 4 metabolic risk factors (1958): Hypertension, Diabetes, hyperlipidemia and obesity (smoking was a non-metabolic factor). The fact that 3 or 4 of these factors often appeared in the same subject was noted in the literature and in 1988 Prof Gerald Reavan from Stanford popularized the term Metabolic Syndrome. The consensus was that insulin resistance was the underlying condition that precipitated the features of the syndrome. Approximately 10 years later work by Graham and Hardie and others showed/implicated that a metabolic switch (AMPK) was central to all the features seen in Metabolic syndrome. AMPK is an energy sensor (enzyme) in all cells that is activated/deactivated when the ratio of ATP/AMP is decreased/increased. When AMPK is activated the body is in catabolic or fat burning mode. Conversely when AMPK is inactive the body is in fat storage mode.

The current clinical thinking in terms of the sequence of events that leads to the metabolic syndrome is likely the following:
over-nutrition or perhaps inappropriate nutrition (high fructose corn syrup as an example to be further elucidated below);
leading to “fatty liver” (AMPK probably plays a central role in preventing or permitting the development of fatty liver),
leading to insulin resistance,
leading to obesity,
leading to diabetes, hypertension, and hyperlipidemia.

The process may be further amplified by an inflammatory component that is generated by nonalcoholic steatohepatitis (“NASH”) or obesity itself.

Separate from the work of AMPK in metabolic syndrome is the contribution of AMPK in improving fitness, endurance and athletic performance. Major pharmaceutical companies have even attempted to create a performance enhancing agonist of AMPK called AICAR. The results were sufficiently compelling that the world anti-doping federation declared it a banned substance.

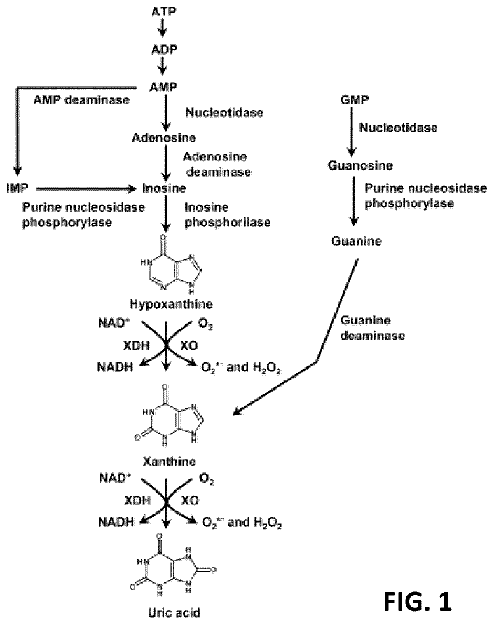

In parallel, elevated uric acid had been associated with all aspects of the metabolic syndrome. The contribution of uric acid in this pathology is not obvious. Uric acid is an end product of purine metabolism (FIG. 1). Endogenous sources of purine are ATP (clearly connecting to the previous discussion of AMPK) and GTP.

Exogenous sources are sugar and beer. Sugar which is disaccharide composed of fructose and glucose. Fructose has the unique property of actually using up or depleting ATP prior to helping create it in the mitochondrial citric acid cycle.

There are two biochemical pathways to generate ATP.

De novo synthesis as a result of using carbohydrates (glycolysis), lipids (fatty oxidation) and amino acids as substrates converging to a common mechanism (tricarboxylic acid cycle) in the mitochondria and resulting in the generation of ATP.

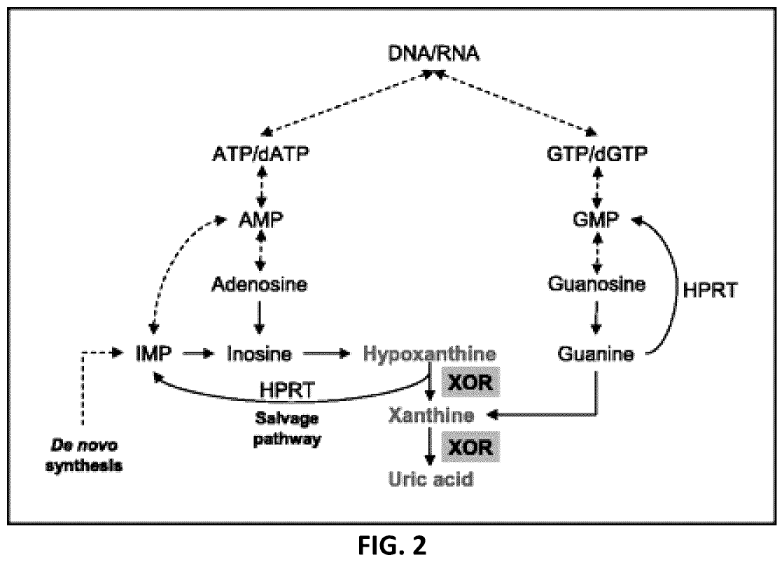

The second pathway is often referred to as the Salvage pathway (FIG. 2). When ATP is used to provide energy in cellular processes it is degraded to ADP then AMP then Inosine and eventually Uric acid.

The salvage pathway refers to the creation of ATP by reversal of the degradation cascade and using the degradation products to build the ATP molecule back up.

Without being limited by any particular theory, when pharmaceutical agents such as Allopurinol or Febuxostat (both Xanthine oxidase inhibitors) are used to block the conversion of hypoxanthine and xanthine to uric acid, there is an increase in cellular ATP. Several studies have shown this result including in clinical studies in humans.

In addition, it is interesting to note that there exists a synergistic effect in increasing ATP in the cell by combining Inosine with Febuxostat. Inosine alone did not affect ATP levels but in combination with Febuxostat increased ATP levels beyond levels of Febuxostat alone. Inosine (as seen below in both diagrams) is a degradation product of ATP and can be used by salvage to make more ATP.

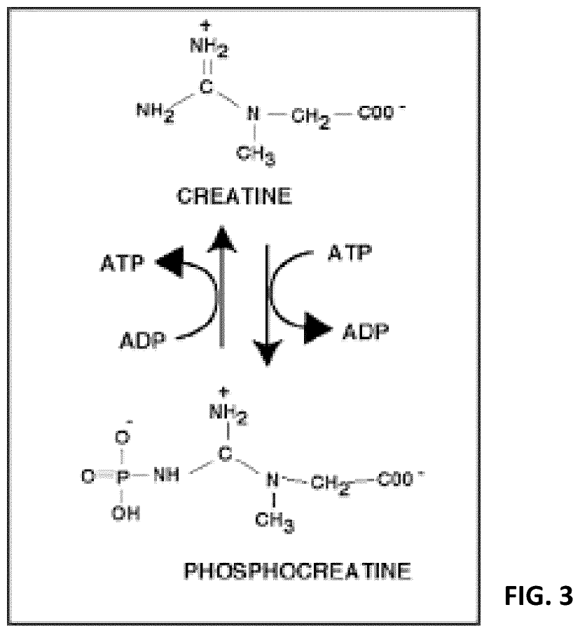

Finally, a last ingredient to compliment ATP production is phosphocreatine. Phosphocreatine facilitates the creation of ATP from ADP (FIG. 3) and is an additional non-overlapping pathway leading to increase cell ATP and thus energy.

A side chain of fructose metabolism actually generates uric acid. The uric acid is believed to indirectly and directly inactive AMPK and therefor contribute to fatty liver and insulin resistance leading to metabolic syndrome. There exist numerous peer reviewed articles that support this thesis including genetic and molecular cell biology as well as clinical trials. Humans and primates are the only mammals that have a high uric acid level and in general the only species that naturally develop metabolic syndrome. All other mammals possess an enzyme, uricase, which dissolves uric acid. Our biological ancestors lost the uricase gene 15 million years ago in the Miocene global cooling. Prior to that these biological ancestors ate fruit as a primary source of food. With global cooling tropical forests dried up as did the food supply. Loss of the uricase gene promoted a high uric acid level permitting fast and efficient fat storage and enabling the species to survive at least a month without food. This conferred a survival advantage when food was scarce but became a disadvantage when nutrition is available 24 hours a day. Thus, giving the basis for the obesity epidemic we now witness where processed foods that are enriched with high fructose corn syrup is heavily used. As an interesting demographic proof of this concept are the Maori of New Zealand who have both the highest incidence of gout (high uric acid) in the world as well as the highest rate of metabolic syndrome (80% of the population).

The medical research that encompasses the discussion above resides in three largely independent domains: 1) uric acid and metabolic syndrome, 2) AMPK and metabolic syndrome, athletic performance, endurance and recovery, and 3) inflammation and its role in metabolic syndrome and recovery.

As a matter of background, “cardio exercise” is known to reduce insulin resistance improve hypertension and also activate AMPK. AMPK induces a long list of cascading events including but not limited to activation of nitric oxide pathways, lipid catabolism and mitochondrial biogenesis.

A standard therapy employed in combating insulin resistance is Metformin (prescription RX). Metformin likely works by disrupting “complex I” of the electron transport chain and indirectly activating AMPK. Other therapeutics have also been shown to influence AMPK. Aspirin activates AMPK, as well as reducing inflammation via the COX pathway. Recently, allopurinol (a xanthine oxidase inhibitor that reduces uric acid levels) has also been shown to affect elements of metabolic syndrome.

For hundreds of years cherries have been used as a natural remedy to treat gout: an arthritic condition that is a result of uric acid crystals in the joint space. Several studies demonstrate that tart cherries can reduce serum levels of uric acid as well as other markers of inflammation such as CRP and sedimentation rate. The biochemistry that underlies the effect is twofold. An inhibition of the enzymes that generate uric acid as well as an anti-inflammatory effect on the COX pathway. The effect is likely due to the concentration of anthocyanins and phenolics in the specific cultivar or type of cherry.

There is a large and separate body of literature that has studied the use of tart cherries as improving endurance, performance and recovery in athletics. The cultivars used are generally speaking Montmorency cherries, known for high anthocyanin and phenolic levels. A large majority of these studies show significant improvement in endurance performance and recovery and some of these studies are double-blinded placebo-controlled crossover designed. Further evidence of the mechanism is that in the few studies that had negative outcomes, the dosage used was insufficient to lower the uric acid.

There are some studies that have tested tart cherries as improving aspects of metabolic health including a recent pilot study showing a lowering of insulin resistance. However even in these studies, the underlying mechanism of uric acid is never invoked.

Without being limited by any particular theory, Tart cherry (Montmorency and Balaton cultivars) are known to have the highest levels of phenolics and anthocyanins. However, the anthocyanin I, II, and Phenolic concentrations have not been fully established.

High anthocyanin, high polyphenolic Tart cherries such as Montmorency and Balaton have been shown to reduce uric acid by inhibiting xanthine oxidase (XO).

Lowering uric acid can increase cellular ATP in 2 ways.
The enzyme AMPK is known as the master metabolic sensor. Active AMPK generates de novo ATP from fat, carbohydrates etc. Uric acid inhibits AMPK. Lowering uric acid modestly nonlinearly increases AMpk activity and thus increase ATP.
Lowering uric acid production by inhibiting XO also increases the Purine salvage pathway which independently increases ATP.

As an additional point of differentiation, Tart Cherry also inhibits COX I and II enzymes that are responsible for generating proinflammatory molecules.

There is a strong relationship between inflammation and cell energetics. The anti-inflammatory component of tart cherries is a positive physiological attribute with respect to energy production and as such can be regarded as having energy benefits beyond other XO inhibitors.

There is a need for a formulation for inhibiting, ameliorating, delaying the onset of, reducing the likelihood of, treating, and/or preventing metabolic syndrome, and/or increasing energy levels.

### Formulations and Methods

The present disclosure is related to formulations for inhibiting, ameliorating, delaying the onset of, reducing the likelihood of, treating, and/or preventing metabolic syndrome, and/or increasing energy levels. Besides metabolic syndrome, the formulations are intended to improve fitness-related end points, including performance, endurance, recovery and/or energy levels. An embodiment of the formulations according to the present disclosure is referred to as Ten Plus. An embodiment of Ten Plus is provided in the form of a dark cherry supplement. An embodiment of Ten Plus is provided in the form of a dark cherry supplement, which provides natural metabolic energy. An embodiment of Ten Plus is provided in the form of a dark cherry supplement, which provides natural metabolic energy, and supports metabolic health. An embodiment of Ten Plus is provided in the form of a dark cherry supplement, which provides natural metabolic energy, and supports weight control. An embodiment of Ten Plus is provided in the form of a dark cherry supplement, which supports metabolic health and weight control. An embodiment of Ten Plus is provided in the form of a dark cherry supplement, which provides natural metabolic energy, and supports metabolic health and weight control. The ingredients of an embodiment of Ten Plus is shown in Table 2:

TABLE 2
SUPPLEMENT FACTS
Serving Size: 1 scoop (4 g)
Servings Per Container: 30

| | Amount | | |
|---|---|---|---|
| | per | | % Daily |
| | Serving | | Value |
| Calories | 10 | | |
| Total Carbohydrate | 3 | g | 1% ** |
| Dietary Fiber | 1 | g | 4%** |
| Protein | | | ** |
| Vitamin C | 250 | mg | 278% |
| Sodium | 5 | mg | <1% |
| Tart Cherry Whole Fruit | 1.5 | g | † |
| Grape Skin Extract 45% Proanthocyanin | 200 | mg | † |
| Turmeric Root Extract (20% Curcumin) | 25 | mg | † |

** Percent Daily Values are based on a 2,000 calorie diet.
† Daily value not established
Other ingredients: Natural Flavor Maltodextrin, Stevia Leaf Extract, Citric Acid, Beet (Color), Silicon Dioxide (Anti-caking agent)

In some embodiments, the formulations comprise at least one/one or more tart cherry extract from Montmorency cherries, Balaton cherries, or a mixture of Montmorency and Balaton cherries. The formulations additionally and/or optionally comprise one or more antioxidant vitamins, one or more polyphenolic compounds with antioxidant and/or anti-inflammatory properties, and one or more prebiotics. In some embodiments, the one or more antioxidant vitamins includes beta-carotene, vitamin C, and vitamin E. In some embodiments, the one or more polyphenolic compounds with antioxidant and/or anti-inflammatory properties include curcumin, resveratrol, flavonoids, flavanols, flavonols, flavonones, flavones, isoflavones, anthocyanins, lignans, stilbenes, and the like. In some embodiments, the resveratrol is obtained from an extract of grape seed, grape seed oil, or grape skin. In some embodiments, the one or more prebiotics include inulin. Non-limiting examples of sources of prebiotic can include chicory root, garlic, dandelion greens, Jerusalem artichoke, onions, leeks, asparagus, bananas, barley, oats, apples, Konjac root, cocoa, Burdock root, flax seeds, Yacon root, jicama root, wheat bran, and seaweed. In some embodiments, the formulations further comprise one or more of inosine, and creatine.

Disclosed herein are embodiments of formulations comprising various combinations of Tart cherry, Vitamin C, Turmeric (Curcumin), Inulin, and Grape seed extract for inhibiting, ameliorating, delaying the onset of, reducing the likelihood of, treating, and/or preventing metabolic syndrome, and/or increasing energy levels. In some embodiments, the formulations comprising various combinations of Tart cherry, Vitamin C, Turmeric (20% Curcumin), Inulin, and Grape seed extract are for inhibiting, ameliorating, delaying the onset of, reducing the likelihood of, treating, and/or preventing metabolic syndrome. In some embodiments, the formulations comprising various combinations of Tart cherry, Vitamin C, Turmeric (20% Curcumin), Inulin, and Grape seed extract are for increasing energy levels. In some embodiments, the formulations comprising various combinations of Tart cherry, Vitamin C, Turmeric (20% Curcumin), Inulin, and Grape seed extract are for both inhibiting, ameliorating, delaying the onset of, reducing the likelihood of, treating, and/or preventing metabolic syndrome, and/or increasing energy levels.

In some embodiments, the formulation is an oral supplement. In some embodiments, the formulation is an edible solid or an edible semi-solid. In some embodiments, the formulation is a potable liquid. In some embodiments, the formulation is a powder or pellet that can be reconstituted in a potable liquid (e.g., water). In some embodiments, the formulation is a tablet or a capsule that can be taken without reconstitution.

Embodiments of the formulations provided herein comprise active ingredients, inactive ingredients, excipients, additives, and/or pharmaceutically acceptable carriers. Examples of additives include natural polymer compounds, inorganic salts, binders, lubricants, disintegrants, surfactants, thickeners, coating agents, pH adjusters, antioxidants, flavoring agents, preservatives, and colorants among others. Examples of other pharmaceutically acceptable carriers include liquid carriers such as water, alcohol, emulsion, and solid carriers such as gel, powder, etc. Standard pharmaceutical formulation techniques and ingredients can be used, such as those disclosed in Remington's The Science and Practice of Pharmacy, 21st Ed., Lippincott Williams & Wilkins (2005), which is hereby incorporated by reference in its entirety.

Embodiments of the formulations for oral administration can be any dosage form that is suitable for oral ingestion, for example, liquid formulations such as elixir, suspension, syrup, emulsion, ampoule, a premixed ready-to-consume drink, etc., solid formulations such as gel, gum, drop, powder, granule, pill, sugar-coated tablet, film-coated tablet, capsule, package agent, etc. Also contemplated are sustained-release formulations such as gel-coated formulations, multi-coated formulations, localized release formulations.

In some embodiments, the formulation is provided in the form of a tablet, capsule, gel cap, or softgel. The tablet, capsule, gel cap, or softgel may comprise additional inactive ingredients such as fillers including, but not limited to, rice flour, methylcellulose, magnesium stearate. Several tablets, capsules, gel caps, or softgels typically comprise one daily dose of each of the active ingredients, as described below.

In some embodiments, the formulation is a single serving formulation to be taken once per day by a subject. Thus, in some embodiments a daily dose of active ingredients is provided in a single serving. In some embodiments one daily dose may be divided among two or more servings to be taken in a day, for example among two or more servings of a liquid beverage or two or more tablets. A daily dose of the formulation can be taken by or administered to a subject in a 24 hour period. In some embodiments, a daily dose of the formulation is taken every day for 1 week to 12 months. In some embodiments, the formulation is taken for 1, 2, 3 or 4 weeks. In some embodiments, the formulation is taken for 2, 3, 4, 5, 6, 7, 8, 9, 10, 11 or 12 months, or a value within a range defined by any two of the aforementioned values.

In some embodiments, a daily dose of the formulation can be provided in one or more daily servings. In some embodiments, a daily dose is provided in a single serving. In some embodiments, a daily dose may be provided in two or more servings (up to 5 servings in 24 hours). While the size of each serving may vary, the quantity of active ingredients to be consumed will be equal to one serving. In the case of a single serving, where one single serving is intended to be taken per day, the single serving will comprise a complete daily dose. In the case of multiple servings, the quantity of active ingredients will be such that the total amount of active ingredients in the servings to be taken in a single day is equivalent to a daily dose.

In some embodiments, the formulation is provided in the form of a liquid drink. The liquid drink may comprise the active ingredients in a water base, or in another base liquid that is inert with respect to the active ingredients. The liquid drink may comprise one or more additional inactive ingredients. In some embodiment the liquid drink may comprise one or more gelling or stabilizing agents including, but not limited, to xanthan gum, guar gum, propylene glycol, acacia gum and maltodextrin. In some embodiments the liquid drink may comprise one or more colorants, for example a natural color such as carmine or beet root. In some embodiments the liquid drink may comprise one or more preservatives such as citrus oil, sorbates and benzoates. The liquid drink may also comprise one or more flavors such as fruit (e.g., strawberry, melon, apple, peach, lemon, orange, mango, and the like) and non-fruit flavors. In some embodiments the flavor is a natural flavor. In some embodiments the liquid drink may comprise artificial and natural sweeteners.

In some embodiments the formulation comprises, but is not limited to, one or more artificial sweeteners such as sucralose, acesulfame K or a combination of these and/or one or more natural sweeteners such as sugar, fructose, honey, erythritol, xylitol, stevia, monk fruit, agave, citrus and protein extracts used as sweeteners or combinations thereof.

In some embodiments, the formulation is a liquid drink comprising a single serving of about 4.5 fl. oz. However, the skilled artisan will appreciate that while the size of the serving itself may vary depending on the quantity of the inactive ingredients, such as water, the amount of the active ingredients in each single serving will be within the ranges provided herein. For example, while the single serving size may be ½ fl. oz, 1 fl. oz, 2 fl. oz., 4 fl. oz., 6 fl. oz., 10 fl. oz., 12 fl. oz., 16 fl. oz. or anything smaller or larger, or a value within a range defined by any two of the aforementioned values, the daily dose of active ingredients will be the same in each serving. In some embodiments a single serving is about 2 fl. oz. to about 54 fl. oz. In some embodiments, the formulation is a liquid concentrate comprising of multiple servings to be diluted into a liquid beverage to obtain a single serving size.

In some embodiments, the formulation is a liquid drink comprising a single serving of about 4.23 fl. oz. (120 grams) (Table 2).

In some embodiments, the formulation comprises one or more additional components that improve absorption of one or more of the active ingredients.

In some embodiments, the formulation is taken in the morning on an empty stomach. In some embodiments, the formulation is taken at any time enhanced energy levels are needed. In some embodiments, the formulation is taken daily to provide enhanced energy levels. In some embodiments, the formulation is taken to promote a feeling of overall well-being.

In some embodiments, general health improvements after taking the formulation may include one or more of inhibition, amelioration, delayed onset of, reduced likelihood of, treatment, and/or prevention of metabolic syndrome, and increase in energy levels. In some embodiments, general health improvements after taking the formulation includes include one or more of inhibition, amelioration, delayed onset of, reduced likelihood of, treatment, and/or prevention of metabolic syndrome. In some embodiments, general health improvements after taking the formulation includes increase in energy levels. In some embodiments, general health improvements after taking the formulation includes inhibition, amelioration, delayed onset of, reduced likelihood of, treatment, and prevention of metabolic syndrome, as well as increase in energy levels.

In some embodiments, general health improvements including, but not limited to, inhibition, amelioration, delayed onset of, reduced likelihood of, treatment, and/or prevention of metabolic syndrome, and/or increase in energy levels are observed within 1 day to about 21 days of taking an embodiment of the formulations herein. In some embodiments, general health improvements are observed within 1 day to about 7 days. In some embodiments, general health improvements are observed within 1 day to about 14 days. In some embodiments, general health improvements are observed within 1 day to about 5 days. In some embodiments, general health improvements are observed within 1 day to about 10 days. In some embodiments, general health improvements are observed within 1 day to about 15 days. In some embodiments, general health improvements are observed within 1 day to about 20 days.

In some embodiments, inhibition, amelioration, delayed onset of, reduced likelihood of, treatment, and/or prevention of metabolic syndrome, and increase in energy levels may also be associated with high levels of concentration, optimal cognitive function, effective decision making, keen comprehension, effortless recall, mental clarity, quick reflexes, restful sleep, increased motivation, coordinated motor function, efficient problem solving, mental acuity, optimal energy levels, overall well-being, ability to focus, mood stability, memory support, alertness, improved neurotransmission, and nerve cell integrity.

In some embodiments, the formulations may provide benefits to subjects suffering from other conditions related to metabolic syndrome and lower energy levels. Non-limiting examples include Post Traumatic Stress Disorder (PTSD), Attention Deficit Disorder (ADD), Attention Deficit Hyperactivity Disorder (ADHD), anxiety, migraine, dementia, tinnitus, and in hangovers.

In some embodiments, formulations comprising combinations of Tart cherry, Vitamin C, Turmeric (20% w/w Curcumin), Inulin, and Grape seed extract are provided. In some embodiments, formulations comprising sub-combinations of Tart cherry, Vitamin C, Turmeric (Curcumin), Inulin, and Grape seed extract are provided. In some embodiments, the combinations and sub-combinations further comprise Inosine. In some embodiments, the combinations and sub-combinations further comprise creatine. In some embodiments, the combinations and sub-combinations further comprise Inosine and creatine.

In some embodiments, the combinations and sub-combinations are intended to target and utilize multiple biochemical pathways and entry points for energy (ATP) production. For example, tart cherry can inhibit Xanthine Oxidase of the salvage pathway, Curcumin, grape seed, and Tart cherry can cause AMPK activation (i.e., activate classical TCA cycle pathway, Inosine can enhance the salvage pathway, Creatine provides extra substrate for final ATP production, and the anti-inflammatory properties tart cherry, grape seed, and curcumin can save and conserve energy.

In some embodiments, the ingredients in the combinations and sub-combinations act synergistically to amplify the effect of formulation in inhibiting, ameliorating, delaying the onset of, reducing the likelihood of, treating, and/or preventing metabolic syndrome, and/or increasing energy levels by affecting more than one physiological targets via independent mechanisms. For example, without being limited by any particular theory, Tart Cherry can lower uric acid levels, as well as indirectly increase AMPK and NO as well as reduce inflammation via cox pathway, Vitamin C can increase excretion of uric acid and anti-oxidant, Curcumin can directly increase AMPK and anti-inflammatory, the anti-oxidant resveratrol in Grape seed extract can act against insulin resistance, and Inulin, a prebiotic, can mitigate metabolic syndrome.

Without being limited by any particular theory, while uric acid's contribution to metabolic syndrome is still emerging, lowering uric acid is for the most part restricted to treating gout (very high levels of uric acid that precipitate an arthritis). In some embodiments, the formulations herein can provide the advantage of lowering uric acid even from the “normal range” to improve metabolic function including normalizing blood pressure, lipids, fat storage, insulin resistance, etc. The amount of lowering uric acid even mildly is likely to induce changes in metabolic function in a far more effective way than the original gout treatment. In some embodiments, the formulations can be used in combination with other drugs that are currently used. As a result, potentially synergistic effects can be achieved. For example, the formulations herein can be combined with metformin, which is one of the treatments for insulin resistance. Lowering uric acid can lower gout. In some embodiments, Vitamin C in the formulation further lowers uric acid by alternative mechanisms and has a synergistic. In some embodiments, Curcumin and grape seed extract further promote metabolic health from alternative pathways and provide synergistic effects. In some embodiments, the anti-inflammatory properties of tart cherry address another concurrent pathological pathway (inflammation) which is connected to obesity. In some embodiments, the formulations herein can be used to improve access to the body's natural energy stores and improve endurance and performance. In some embodiments, the additional anti-inflammatory properties of the formulations disclosed herein can be also used in recovery from exercise or injury. In some embodiments, the formulations can be used as an energy and health supplement.

In some embodiments, the formulation comprises at least 4 active ingredients that are synergistic in their effects. In some embodiments, the formulation comprises at least 4, 5, 6, 7, 8, 9, 10, 11, 12 or 13 synergistic active ingredients, or a value within a range defined by any two of the aforementioned values. For example, the active ingredients may be synergistic with respect to their positive effects with respect to inhibiting, ameliorating, delaying the onset of, reducing the likelihood of, treating, and/or preventing metabolic syndrome, and/or increasing energy levels.

In some embodiments, the effects of 4 or more of the active ingredients are additive. In some embodiments, the effects of at least 4, 5, 6, 7, 8, 9, 10, 11, 12 or 13 of the active ingredients are additive, or a value within a range defined by any two of the aforementioned values. For example, the effects may be additive with respect to having positive effects with respect to inhibiting, ameliorating, delaying the onset of, reducing the likelihood of, treating, and/or preventing metabolic syndrome, and/or increasing energy levels.

In some embodiments, the effects of the at least 4-13 active ingredients are additive. In some embodiments, the effects of the at least 4-13 active ingredients are synergistic. In some embodiments, the effects of the at least 4-13 active ingredients are additive and/or synergistic.

In some embodiments, the formulations comprise about 1500 mg of Tart cherry. In some embodiments, the formulations comprise about 250 mg to about 2500 mg of Tart cherry. In some embodiments, the formulations comprise about 500 mg to about 5000 mg of Tart cherry. In some embodiments, the formulations comprise about 750 mg to about 7500 mg of Tart cherry. In some embodiments, the formulations comprise about 1000 mg to about 15000 mg of Tart cherry.

In some embodiments, the formulations comprise about 250 mg of Vitamin C. In some embodiments, the formulations comprise about 25 mg to about 500 mg of Vitamin C. In some embodiments, the formulations comprise about 100 mg to about 1000 mg of Vitamin C. In some embodiments, the formulations comprise about 200 mg to about 2000 mg of Vitamin C. In some embodiments, the formulations comprise about 500 mg to about 2500 mg of Vitamin C.

In some embodiments, the formulations comprise about 25 mg Turmeric (20% w/w Curcumin). In some embodiments, the formulations comprise about 2.5 mg to about 10 mg Turmeric (20% w/w Curcumin). In some embodiments, the formulations comprise about 5 mg to about 50 mg Turmeric (20% w/w Curcumin). In some embodiments, the formulations comprise about 25 mg to about 125 mg Turmeric (20% w/w Curcumin). In some embodiments, the formulations comprise about 100 mg to about 250 mg Turmeric (20% w/w Curcumin).

In some embodiments, the formulations comprise about 500 mg of Inulin. In some embodiments, the formulations comprise about 50 mg to about 350 mg of Inulin. In some embodiments, the formulations comprise about 300 mg to about 1500 mg of Inulin. In some embodiments, the formulations comprise about 2000 mg to about 3500 mg of Inulin. In some embodiments, the formulations comprise about 3500 mg to about 5000 mg of Inulin.

In some embodiments, the formulations comprise about 200 mg Grape seed extract. In some embodiments, the formulations comprise about 20 mg to about 500 mg Grape seed extract. In some embodiments, the formulations comprise about 250 mg to about 750 mg Grape seed extract. In some embodiments, the formulations comprise about 500 mg to about 1500 mg Grape seed extract. In some embodiments, the formulations comprise about 1000 mg to about 2000 mg Grape seed extract.

In some embodiments, the formulation comprises the following active ingredients in the indicated recommended amounts in one dose or serving of the formulation, for example in a daily dose:
Tart cherry extract—about 0.25 gm/day to about 5 gm/day. Without being limited by any particular theory, this dosage is equivalent to about 180 Montmorency cherries.
Vitamin C—0 gm/day to about 2 gm/day
Curcumin—0 gm/day to about 2 gm/day.
Grape seed oil extract—about 0 mg/day to about 300 mg/day.
Inulin—0 gm/day to about 10 gm/day.

In some embodiments, the formulations comprise Tart cherry extract at a dose range of about 0.25 gm to about 5 gm per daily dose or serving.

In some embodiments, the formulations comprise Vitamin C at a dose range of about 0 gm to about 2 gm per daily dose or serving.

In some embodiments, the formulations comprise Curcumin at a dose range of about 0 gm/to about 2 gm per daily dose or serving.

In some embodiments, the formulations comprise Grape seed oil extract at a dose range of about 0 mg to about 300 mg per daily dose or serving.

In some embodiments, the formulations comprise Inulin at a dose range of about 0 gm to about 10 gm per daily dose or serving.

In some embodiments of the formulation, an effective amount of the at least one extract from a tart cherry ranges from about 1.5 grams to about 2.5 grams. In some embodiments of the formulation, an effective amount of the at least one extract from a tart cherry ranges from about 0.15 gram to about 25 grams.

In some embodiments of the formulation, an effective amount of the at least one antioxidant vitamin ranges from about 250 mg to about 500 mg. In some embodiments of the formulation, an effective amount of the at least one antioxidant vitamin ranges from about 25 mg to about 5000 mg.

In some embodiments of the formulation, an effective amount of the at least one polyphenolic compound ranges from about 25 mg to about 200 mg. In some embodiments of the formulation, an effective amount of the at least one polyphenolic compound ranges from about 2.5 mg to about 2000 mg.

In some embodiments of the formulation, an effective amount of the at least one prebiotic is about 450 mg. In some embodiments of the formulation, an effective amount of the at least one prebiotic ranges from about 45 mg to about 4500 mg.

In some embodiments, an effective amount of Tart cherry extract in the formulation ranges from about 0.15 gram to about 25 grams. In some embodiments, an effective amount of Tart cherry extract in the formulation ranges from about 1.5 grams to about 2.5 grams.

In some embodiments, an effective amount of Vitamin C in the formulation ranges from about 25 mg to about 5000 mg. In some embodiments, an effective amount of Vitamin C in the formulation ranges from about 250 mg to about 500 mg.

In some embodiments, an effective amount of Grape seed extract in the formulation ranges from about 20 mg to about 2000 mg. In some embodiments, an effective amount of Grape seed extract is about 200 mg.

In some embodiments, an effective amount of Turmeric in the formulation ranges from about 2.5 mg to about 250 mg. In some embodiments, an effective amount of Turmeric in the formulation is about 25 mg.

In some embodiments, an effective amount of Inulin in the formulation ranges from about 45 mg to about 4500 mg. In some embodiments, an effective amount of Inulin in the formulation is about 450 mg.

Any one or more of the embodiments of the formulations and kits of formulations disclosed herein can be used in methods for inhibiting, ameliorating, delaying the onset of, reducing the likelihood of, treating, and/or preventing metabolic syndrome, and/or increasing energy levels.

Any one or more of the embodiments of the formulations and kits of formulations disclosed herein can be used in methods for inhibiting, ameliorating, delaying the onset of, reducing the likelihood of, treating, and/or preventing metabolic syndrome.

Any one or more of the embodiments of the formulations and kits of formulations disclosed herein can be used in methods for increasing energy levels.

### Methods of Making Formulations

Also disclosed herein are embodiments of methods of making any of the formulations disclosed herein.

In some embodiments, methods are described for making the embodiments of the formulations herein, wherein the formulations comprise one or more ingredients that are prone to floatation, sedimentation, and/or precipitation. In some embodiments, methods of making a liquid dietary supplement drink using a combination of synergistic ingredients that target long and short-term human brain health are described in which the formation of floaties (precipitate) or bottom-settlement (sediment) in the formulation is reduced or avoided entirely.

In some embodiments, the generation of a sediment and/or precipitate can be reduced or avoided by combining the ingredients in one or more particular sequences. Thus, in some embodiments special attention is given to the order in which each ingredient is added. In particular, in some embodiments the ingredients are added in an order which results in all of the ingredients going into solution, or staying in suspension.

In addition to avoiding precipitation and sedimentation, other indicators of stability of the formulation can be preserved during preparation of the formulation. These indicators of stability of the formulation may include, for example, flavor profile, texture and mouth-feel. Thus, in some embodiments, one or more indicator of stability, such as flavor profile, texture and mouth-feel of the formulation prepared according to the methods described herein are substantially as desired.

In some embodiments, the ingredients are freeze dried to create a powder that minimizes loss of potency before preparing a formulation.

In some embodiments, any of the ingredients in the formulations can be the first ingredient to be added to water or other liquid in preparing a liquid formulation. The remaining ingredients may be added in any order or combination. Flavor components, colorants and sweeteners, if any, are typically added last.

In some embodiments, tart cherry extract is the first ingredient that is added to water or other liquid in preparing a liquid formulation. The remaining ingredients may be added in any order or combination.

In some embodiments, Vitamin C is the first ingredient that is added to water or other liquid in preparing a liquid formulation. The remaining ingredients may be added in any order or combination.

In some embodiments, Curcumin is the first ingredient that is added to water or other liquid in preparing a liquid formulation. The remaining ingredients may be added in any order or combination.

In some embodiments, grape se extract is the first ingredient that is added to water or other liquid in preparing a liquid formulation. The remaining ingredients may be added in any order or combination.

In some embodiments, inulin is the first ingredient that is added to water or other liquid in preparing a liquid formulation. The remaining ingredients may be added in any order or combination.

In some embodiments, the formulation is prepared in the form of a liquid drink. The liquid drink may comprise the active ingredients in a water base, or in another base liquid that is inert with respect to the active ingredients. The liquid drink may comprise one or more additional inactive ingredients. In some embodiments the liquid drink may comprise one or more preservatives such as citrus oil, sorbates and benzoates. The liquid drink may also comprise one or more flavors such as fruit (e.g., strawberry, melon, apple, peach, lemon, orange, mango, and the like) and non-fruit flavors. In some embodiments the flavor is a natural flavor.

In some embodiments, sweeteners, such as sucralose or acesulfame K or a combination thereof, are added. In some embodiments, a natural sweetener such as sugar, fructose, honey, agave, monk fruit, stevia, erythritol, xylitol, citrus and protein extracts or a combination thereof are added.

In some embodiments, a liquid formulation is prepared by combining the following water, tart cherry extract, vitamin, curcumin, grape seed extract, and inulin. In some embodiments, inosine is optionally added. In some embodiments, creatine is optionally added. In some embodiments, inosine and creatine are optionally added.

In some embodiments, the ingredients are mixed in an order that allows for optimum solubility throughout the liquid phase. In some embodiments, the ingredients are mixed in an order that does not interfere with the dispersion and solubility of other ingredients throughout the liquid phase and does not, for example, lead to clumps (precipitation) and/or fall-out (sedimentation) throughout the liquid. In some embodiments, the ingredients are mixed in a specific order that is empirically determined and that allows for optimum solubility throughout the liquid phase. In some embodiments, the ingredients are mixed in an order that is empirically determined and that does not interfere with the dispersion and solubility of other ingredients throughout the liquid phase and does not, for example, lead to clumps (precipitation) and/or fall-out (sedimentation) throughout the liquid.

In some embodiments, one or more additional ingredients are added to water one or more premixes, each premix comprising a fraction of the other ingredients. The premixes may be added in multiple steps or in a single step.

In some embodiments, all additional ingredients are added to water as a single premix comprising all the additional ingredients.

In some embodiments, the time required for dissolving and/or dispersing the ingredients in water at least about 10 to about 60 minutes. In some embodiments, the time required is about 10 to about 30 minutes. In some embodiments, the time required is about 20 to about 60 minutes.

In some embodiments, additional flavoring components are added to the formulation. In some embodiments, any and all flavoring components, such as Natural flavors, and sweeteners are added next, either individually and sequentially any order, or simultaneously. Exemplary sweeteners include, but are not limited to natural sweeteners, such as Sugar, Fructose, Honey, Agave, Monk fruit, Stevia, Erythritol, Xylitol, Citrus and Protein extracts, and artificial sweeteners such as Sucralose, Acesulfame K, or a combination of these.

The ingredients and flavoring components may be dissolved in the solution, for example, by one or more of the following techniques: gentle stirring, gentle vortexing, gentle heating. In some embodiments, the time required for dissolving these components is at least about 5 to about 60 minutes. In some embodiments, the time required is about 5 to about 30 minutes. In some embodiments, the time required is about 30 to about 60 minutes.

In some embodiments colorant or colorants are added next and dissolved in the solution, for example, by one or more of the following techniques: gentle stirring, gentle vortexing, gentle heating. In some embodiments, the time required for dissolving these components is at least about 5 to 20 minutes. In some embodiments, the time required is about 30 minutes to about 50 minutes.

In some embodiments one or more salts are added next and dissolved in the solution, for example, by one or more of the following techniques: gentle stirring, gentle vortexing, gentle heating. In some embodiments, the time required for dissolving the one or more salts is at least about 5 to 20 minutes. In some embodiments, the time required is about 30 minutes to about 50 minutes.

In some embodiments, the pH of the finished product is acidic. In some embodiments, the pH of the finished product is about 3 to about 6.5. In some embodiments, the pH of the finished product is about 3.0, 3.5, 4.0, 4.5, 5.0, 5.5, 6.0, or 6.5, or a value within a range defined by any two of the aforementioned values.

In some embodiments, the formulation comprises at least 4 active ingredients that are synergistic in their effects. In some embodiments, the formulation comprises at least 4, 5, 6, 7, 8, 9, 10, 11, 12 or 13 synergistic active ingredients, or a value within a range defined by any two of the aforementioned values. For example, the active ingredients may be synergistic with respect to their positive effects with respect to inhibiting, ameliorating, delaying the onset of, reducing the likelihood of, treating, and/or preventing metabolic syndrome, and/or increasing energy levels.

In some embodiments, the methods result in formulations that comprise about 1500 mg of Tart cherry. In some embodiments, the methods result in formulations that about 250 mg to about 2500 mg of Tart cherry. In some embodiments, the methods result in formulations that about 500 mg to about 5000 mg of Tart cherry. In some embodiments, the methods result in formulations that comprise about 750 mg to about 7500 mg of Tart cherry. In some embodiments, the formulations comprise about 1000 mg to about 15000 mg of Tart cherry.

In some embodiments, the methods result in formulations that comprise about 250 mg of Vitamin C. In some embodiments, the methods result in formulations that comprise about 25 mg to about 500 mg of Vitamin C. In some embodiments, the methods result in formulations that comprise about 100 mg to about 1000 mg of Vitamin C. In some embodiments, the methods result in formulations that comprise about 200 mg to about 2000 mg of Vitamin C. In some embodiments, the methods result in formulations that comprise about 500 mg to about 2500 mg of Vitamin C.

In some embodiments, the methods result in formulations that comprise about 25 mg Turmeric (20% w/w Curcumin). In some embodiments, the methods result in formulations that comprise about 2.5 mg to about 10 mg Turmeric (20% w/w Curcumin). In some embodiments, the methods result in formulations that comprise about 5 mg to about 50 mg Turmeric (20% w/w Curcumin). In some embodiments, the methods result in formulations that comprise about 25 mg to about 125 mg Turmeric (20% w/w Curcumin). In some embodiments, the methods result in formulations that comprise about 100 mg to about 250 mg Turmeric (20% w/w Curcumin).

In some embodiments, the methods result in formulations that comprise about 500 mg of Inulin. In some embodiments, the methods result in formulations that comprise about 50 mg to about 350 mg of Inulin. In some embodiments, the methods result in formulations that comprise about 300 mg to about 1500 mg of Inulin. In some embodiments, the methods result in formulations that comprise about 2000 mg to about 3500 mg of Inulin. In some embodiments, the methods result in formulations that comprise about 3500 mg to about 5000 mg of Inulin.

In some embodiments, the methods result in formulations that comprise about 200 mg Grape seed extract. In some embodiments, the methods result in formulations that comprise about 20 mg to about 500 mg Grape seed extract. In some embodiments, the methods result in formulations that comprise about 250 mg to about 750 mg Grape seed extract. In some embodiments, the methods result in formulations that comprise about 500 mg to about 1500 mg Grape seed extract. In some embodiments, the methods result in formulations that comprise about 1000 mg to about 2000 mg Grape seed extract.

In some embodiments, the methods result in a formulation that comprises the active ingredients listed in Table 0.1 in the indicated recommended amounts in one dose or serving of the formulation, for example in a daily dose:

TABLE 0.1

| Active ingredient | Amounts in one dose or serving |
|---|---|
| Tart cherry extract | about 0.25 gm/day to about 5 gm /day. Without |
| | being limited by any particular theory, this dosage |
| | is equivalent to about 180 Montmorency cherries. |
| Vitamin C | 0 gm/day to about 2 gm/day |
| Curcumin | 0 gm/day to about 2 gm/day. |
| Grape seed oil extract | about 0 mg/day to about 300 mg/day. |
| Inulin | 0 gm/day to about 10 gm/day. |

In some embodiments, the methods result in a formulation that comprises the active ingredients listed in Table 0.2 in the indicated recommended amounts in one dose or serving of the formulation, for example in a daily dose:

TABLE 0.2

| Active ingredient | Amounts in one dose or serving |
|---|---|
| Tart cherry extract | 0.25 gm/day to 5 gm /day. Without being limited |
| | by any particular theory, this dosage is equivalent |
| | to about 180 Montmorency cherries. |
| Vitamin C | 0 gm/day to 2 gm/day |
| Curcumin | 0 gm/day to 2 gm/day. |
| Grape seed oil | 0 mg/day to 300 mg/day. |
| extract | |
| Inulin | 0 gm/day to 10 gm/day. |

In some embodiments, the methods result in formulations that comprise Tart cherry extract at a dose range of about 0.25 gm to about 5 gm per daily dose or serving. In some embodiments, the methods result in formulations that comprise Tart cherry extract at a dose range of 0.25 gm to 5 gm per daily dose or serving.

In some embodiments, the methods result in formulations that comprise Vitamin C at a dose range of about 0 gm to about 2 gm per daily dose or serving. In some embodiments, the methods result in formulations that comprise Vitamin C at a dose range of about 0 gm to 2 gm per daily dose or serving.

In some embodiments, the methods result in formulations that comprise Curcumin at a dose range of about 0 gm/to about 2 gm per daily dose or serving. In some embodiments, the methods result in formulations that comprise Curcumin at a dose range of about 0 gm/to 2 gm per daily dose or serving.

In some embodiments, the methods result in formulations that comprise Grape seed oil extract at a dose range of about 0 mg to about 300 mg per daily dose or serving. In some embodiments, the methods result in formulations that comprise Grape seed oil extract at a dose range of 0 mg to 300 mg per daily dose or serving.

In some embodiments, the methods result in formulations that comprise Inulin at a dose range of about 0 gm to about 10 gm per daily dose or serving. In some embodiments, the methods result in formulations that comprise Inulin at a dose range of 0 gm to 10 gm per daily dose or serving.

In some embodiments the methods result in a formulation that is a single serving to be taken once per day by a subject. Thus, in some embodiments a daily dose of active ingredients is provided in a single serving. In some embodiments one daily dose may be divided among two or more servings to be taken in a day, for example among two or more servings of a liquid beverage or two or more tablets. A daily dose of the formulation is preferably taken by or administered to a subject in a 24-hour period. In some embodiments, a daily dose of the formulation is taken every day for 1 week to 12 months. In some embodiments, the formulation is taken for 1, 2, 3 or 4 weeks. In some embodiments, the formulation is taken for 2, 3, 4, 5, 6, 7, 8, 9, 10, 11 or 12 months, or a value within a range defined by any two of the aforementioned values.

In some embodiments, the methods result in a formulation that is to be taken in the morning on an empty stomach. In some embodiments, the methods result in a formulation that is to be taken at any time mental focus and clarity is needed. In some embodiments, the methods result in a formulation that is to be taken daily to promote brain health. In some embodiments, the methods result in a formulation that is to be taken to promote restful sleep. In some embodiments, the methods result in a formulation that is to be taken to promote a feeling of overall well-being.

In some embodiments, the methods result in a liquid drink formulation such that a single serving of the liquid drink formulation comprises about 4.5 fl. oz. However, the skilled artisan will appreciate that while the size of the serving itself may vary depending on the quantity of the inactive ingredients, such as water, the amount of the active ingredients in each single serving will be within the ranges provided herein. For example, while the single serving size may be ½ fl. oz., 1 fl. oz., 2 fl. oz., 4 fl. oz., 6 fl. oz., 10 fl. oz., 12 fl. oz., 16 fl. oz. or anything smaller or larger, or a value within a range defined by any two of the aforementioned values, the daily dose of active ingredients will be the same in each serving. In some embodiments a single serving is about 2 fl. oz. to about 54 fl. oz. In some embodiments, the formulation is a liquid concentrate comprising of multiple servings to be diluted into a liquid beverage to obtain a single serving size.

In some embodiments, the methods result in a liquid drink formulation comprising a single serving of about 4.23 fl. oz. (120 grams) (Table 2).

In some embodiments of the methods, the product is cold filled. In some embodiments of the methods, the product is pasteurized. In some embodiments of the methods, the product is pasteurized and/or cold filled.

### Kits of Formulations

In some embodiments, a kit is provided which comprises a formulation or combination or subcombination according to any of the embodiments disclosed herein and a dietary plan comprising instructions to use of the formulation in the kit for inhibiting, ameliorating, delaying the onset of, reducing the likelihood of, treating, and/or preventing metabolic syndrome, and/or increasing energy levels. In some embodiments, the dietary plan comprises taking at least one serving of a formulation provided herein per day, for example in the form of a liquid beverage. In some embodiments, the dietary plan can comprise taking at least one serving of a formulation provided herein per day, for example in the form of a liquid beverage of about 4.5 fl. oz. In some embodiments, at least one serving can be taken in the morning on an empty stomach. In some embodiments, at least one serving can be taken at any time, for example, when increased energy levels are desired (e.g., during a workout, during a marathon, etc.). In some embodiments, at least two servings can be taken at any time. In some embodiments, a maximum of five servings in a 24-hour period can be taken. In some embodiments, at least 1, 2, 3, 4, or 5 servings can be taken at any time, or a value within a range defined by any two of the aforementioned values. In some embodiments, the servings are effective in inhibiting, ameliorating, delaying the onset of, reducing the likelihood of, treating, and/or preventing metabolic syndrome, and/or increasing energy levels.

In some embodiments, the kit comprises a formulation in the kit is an edible solid or an edible semi-solid. In some embodiments, the formulation in the kit is a potable liquid. In some embodiments, the formulation in the kit is a powder or pellet that can be reconstituted in a potable liquid (e.g., water). In some embodiments, the formulation in the kit is a tablet or a capsule that can be taken without reconstitution.

### GLP-1 in Obesity and Diabetes

Glucagon-like peptide-1 (GLP-1) is a 30- or 31-amino-acid-long peptide hormone deriving from the tissue-specific posttranslational processing of the proglucagon peptide. It is produced and secreted by intestinal enteroendocrine L-cells and certain neurons within the nucleus of the solitary tract in the brainstem upon food consumption. The initial product GLP-1 (1-37) is susceptible to amidation and proteolytic cleavage, which gives rise to the two truncated and equipotent biologically active forms, GLP-1 (7-36) amide and GLP-1 (7-37). Active GLP-1 protein secondary structure includes two α-helices from amino acid position 13-20 and 24-35 separated by a linker region.

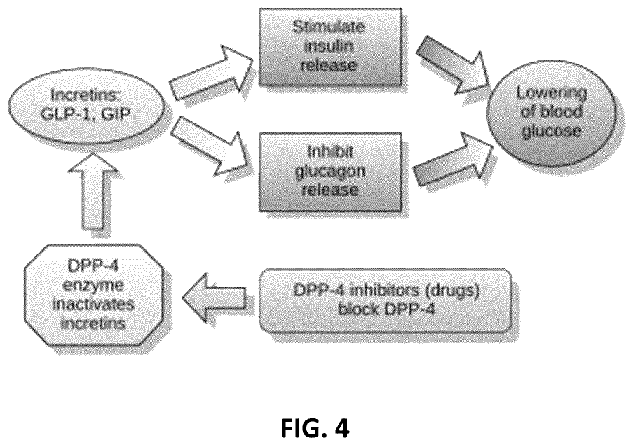

Alongside glucose-dependent insulinotropic peptide (GIP), GLP-1 is an incretin, which has the ability to decrease blood sugar levels in a glucose-dependent manner by enhancing the secretion of insulin (FIG. 4). Beside the insulinotropic effects, GLP-1 has been associated with numerous regulatory and protective effects. Unlike GIP, the action of GLP-1 is preserved in patients with type 2 diabetes. Glucagon-like peptide-1 receptor agonists have gained approval as drugs to treat diabetes and obesity.

In general, when food is consumed, the meal traverses through the stomach where digestion begins and eventually reaches the ileum of the small intestine. The L-cells of the ileum secrete GLP-1 into the blood stream when sensing the absorbed meal. GLP-1 serves as a feedback mechanism for several important physiological functions, including signaling by GLP-1 to the stomach to slow the transit of food and signaling by GLP-1 to the brain resulting in appetite suppression.

Endogenous GLP-1 is rapidly degraded primarily by dipeptidyl peptidase-4 (DPP-4), as well as neutral endopeptidase 24.11 (NEP 24.11) and renal clearance, resulting in a half-life of approximately 2 minutes. Consequently, only 10-15% of GLP-1 reaches circulation intact, leading to fasting plasma levels of only 0-15 pmol/L. Thus, a central challenge in developing a natural GLP-1 supplement is that GLP-1 quickly disappears from circulation with a ½ life of approximately 2 minutes. This degradation is largely due to the enzyme dipeptidyl peptidase-4 (DPP4). To overcome this, GLP-1 receptor agonists and DPP-4 inhibitors have been developed to prolong and increase GLP-1 activity. As opposed to common treatment agents such as insulin and sulphonylurea, GLP-1-based treatment has been associated with weight loss and a lower risk of hypoglycemia, two important considerations for patients with type 2 diabetes.

The normal range for GLP-1 levels in human plasma varies depending on the time of day and metabolic state. For example, in a healthy person, fasting plasma concentration of GLP-1 is between 0 and 15 pmol/L. After a meal, GLP-1 levels increase 2-3 times, reaching a peak within two hours. The peak level can be as high as 60 pmol/L. Oral glucose is a strong stimulus for GLP-1 release. GLP-1 levels are affected by the size and composition of the meal. Certain nutrients, like fatty acids, essential amino acids, and dietary fiber, can also stimulate GLP-1 secretion.

L-arginine, which is an amino acid known to stimulate natural GLP-1 secretion, is an excellent candidate for a non-drug GLP-1 supplement.

The inventors of the present application have developed formulations that overcomes the limitations associated with the short half-lives of GLP-1 and L-arginine. The inventors of the present application have developed formulations that increase GLP-1 levels. The formulations are useful for promoting/enhancing weight loss and is useful in inhibiting, ameliorating, delaying the onset of, reducing the likelihood of, treating, and/or preventing obesity. The formulations are also useful in managing/maintaining/regulating blood sugar levels and is useful in inhibiting, ameliorating, delaying the onset of, reducing the likelihood of, treating, and/or preventing diabetes.

Described herein are formulations comprising L-arginine dosage known to raise GLP-1 physiologically, resveratrol at a dosage that inhibits DPP4 and prolongs GLP-1 half-life, tart cherry powder that contains multiple components known to inhibit arginase and lengthen the presence of arginine in the body. U.S. Pat. No. 11,541,904, which is owned by the current applicant, and which is hereby incorporated by reference in its entirety, applies to the dosage amounts of tart cherry and Vitamin C in this formulation and permits additional items such as L-arginine and resveratrol to be added in the formulation.

The normal range for GLP-1 levels in human plasma vary depending on the time of day and metabolic state. For example, in a healthy person, fasting plasma concentration of GLP-1 is between 0 and 15 pmol/L. After a meal, GLP-1 levels increase 2-3 times, reaching a peak within two hours. The peak level can be as high as 60 pmol/L. Oral glucose is a strong stimulus for GLP-1 release. GLP-1 levels are affected by the size and composition of the meal. Certain nutrients, like fatty acids, essential amino acids, and dietary fiber, can also stimulate GLP-1 secretion.

In some embodiments, GLP-1 levels are increased about 2-fold to about 4-fold. In some embodiments, GLP-1 levels are increased about 3-fold to about 6-fold. In some embodiments, GLP-1 levels are increased about 4-fold to about 8-fold. In some embodiments, GLP-1 levels are increased about 5-fold to about 10-fold. In some embodiments, GLP-1 levels are increased about 2, 3, 4, 5, 6, 7, 8, 9, or 10-fold, or a value within a range defined by any two of the aforementioned values.

In some embodiments, GLP-1 levels are increased 2-fold to 4-fold. In some embodiments, GLP-1 levels are increased 3-fold to 6-fold. In some embodiments, GLP-1 levels are increased 4-fold to 8-fold. In some embodiments, GLP-1 levels are increased 5-fold to 10-fold. In some embodiments, GLP-1 levels are increased 2, 3, 4, 5, 6, 7, 8, 9, or 10-fold, or a value within a range defined by any two of the aforementioned values.

In some embodiments, GLP-1 levels are increased to about 20 to about 35 pmol/L. In some embodiments, GLP-1 levels are increased to about 35 to about 50 pmol/L. In some embodiments, GLP-1 levels are increased to about 50 to about 65 pmol/L. In some embodiments, GLP-1 levels are increased to about 65 to about 80 pmol/L. In some embodiments, GLP-1 levels are increased to about 60 to about 70 pmol/L. In some embodiments, GLP-1 levels are increased to about 70 to about 80 pmol/L. In some embodiments, GLP-1 levels are increased to about 80 to about 90 pmol/L. In some embodiments, GLP-1 levels are increased to about 20, 35, 30, 35, 40, 45, 50, 55, 60, 65, 70, 75, 80, 85, 90, 95, 100, 105, 110, 115, 120, 125, 130, 135, 140, 145, or 150 pmol/L, or a value within a range defined by any two of the aforementioned values.

In some embodiments, increased GLP-1 levels are maintained for about 1 hour to about 24 hours. In some embodiments, increased GLP-1 levels are maintained for about 1 hour to about 4 hours. In some embodiments, increased GLP-1 levels are maintained for about 4 hours to about 8 hours. In some embodiments, increased GLP-1 levels are maintained for about 8 hours to about 12 hours. In some embodiments, increased GLP-1 levels are maintained for about 12 hours to about 16 hours. In some embodiments, increased GLP-1 levels are maintained for about 16 hours to about 20 hours. In some embodiments, increased GLP-1 levels are maintained for about 20 hours to about 24 hours.

In some embodiments, increased GLP-1 levels are maintained for 1 hour to 24 hours. In some embodiments, increased GLP-1 levels are maintained for 1 hour to 4 hours. In some embodiments, increased GLP-1 levels are maintained for 4 hours to 8 hours. In some embodiments, increased GLP-1 levels are maintained for 8 hours to 12 hours. In some embodiments, increased GLP-1 levels are maintained for 12 hours to 16 hours. In some embodiments, increased GLP-1 levels are maintained for 16 hours to 20 hours. In some embodiments, increased GLP-1 levels are maintained for 20 hours to 24 hours.

In some embodiments, the half-life of GLP-1 is increased to about 4 minutes to about 60 minutes. In some embodiments, the half-life of GLP-1 is increased to about 4 minutes to about 10 minutes. In some embodiments, the half-life of GLP-1 is increased to about 10 minutes to about 15 minutes. In some embodiments, the half-life of GLP-1 is increased to about 15 minutes to about 20 minutes. In some embodiments, the half-life of GLP-1 is increased to about 20 minutes to about 25 minutes. In some embodiments, the half-life of GLP-1 is increased to about 25 minutes to about 30 minutes. In some embodiments, the half-life of GLP-1 is increased to about 30 minutes to about 35 minutes. In some embodiments, the half-life of GLP-1 is increased to about 35 minutes to about 40 minutes. In some embodiments, the half-life of GLP-1 is increased to about 40 minutes to about 45 minutes. In some embodiments, the half-life of GLP-1 is increased to about 45 minutes to about 50 minutes. In some embodiments, the half-life of GLP-1 is increased to about 50 minutes to about 55 minutes. In some embodiments, the half-life of GLP-1 is increased to about 55 minutes to about 60 minutes.

In some embodiments, the half-life of GLP-1 is increased to 4 minutes to 60 minutes. In some embodiments, the half-life of GLP-1 is increased to 4 minutes to 10 minutes. In some embodiments, the half-life of GLP-1 is increased to 10 minutes to 15 minutes. In some embodiments, the half-life of GLP-1 is increased to 15 minutes to 20 minutes. In some embodiments, the half-life of GLP-1 is increased to 20 minutes to 25 minutes. In some embodiments, the half-life of GLP-1 is increased to 25 minutes to 30 minutes. In some embodiments, the half-life of GLP-1 is increased to 30 minutes to 35 minutes. In some embodiments, the half-life of GLP-1 is increased to 35 minutes to 40 minutes. In some embodiments, the half-life of GLP-1 is increased to 40 minutes to 45 minutes. In some embodiments, the half-life of GLP-1 is increased to 45 minutes to 50 minutes. In some embodiments, the half-life of GLP-1 is increased to 50 minutes to 55 minutes. In some embodiments, the half-life of GLP-1 is increased to 55 minutes to 60 minutes.

In some embodiments, increasing GLP-1 levels is useful for inhibiting, ameliorating, delaying the onset of, reducing the likelihood of, treating, and/or preventing obesity. In some embodiments, increasing GLP-1 levels is useful is for promoting/enhancing weight loss. In some embodiments, increasing GLP-1 levels is useful for inhibiting, ameliorating, delaying the onset of, reducing the likelihood of, treating, and/or preventing diabetes. In some embodiments, increasing GLP-1 levels is useful for managing/maintaining/regulating blood sugar levels.

In some embodiments, the formulation comprises L-arginine, resveratrol, and at least one extract from a tart cherry. L-arginine increases secretion of natural GLP-1, resulting in an increase GLP-1 levels. Resveratrol inhibits DPP4 thereby preventing degradation of GLP-1 by DPP-4 thereby prolonging the half-life of GLP-1 and further increasing GLP-1 levels. Tart cherry extract contains multiple components that inhibit arginase, which maintains/enhances levels of L-arginine, which in turn increases natural secretion of GLP-1, thereby further increasing GLP-1 levels. Overall, the combination of L-arginine, resveratrol, and tart cherry act synergistically to increase GLP-1 levels, and therefore, is useful in inhibiting, ameliorating, delaying the onset of, reducing the likelihood of, treating, and/or preventing diabetes and obesity.

In some embodiments, the formulation comprises effective amounts of L-arginine, resveratrol, and at least one extract from a tart cherry for increasing GLP-1 levels for inhibiting, ameliorating, delaying the onset of, reducing the likelihood of, treating, and/or preventing obesity. In some embodiments, the formulation comprises effective amounts of L-arginine, resveratrol, and at least one extract from a tart cherry for increasing GLP-1 levels for inhibiting obesity. In some embodiments, the formulation comprises effective amounts of L-arginine, resveratrol, and at least one extract from a tart cherry for increasing GLP-1 levels for ameliorating obesity. In some embodiments, the formulation comprises effective amounts of L-arginine, resveratrol, and at least one extract from a tart cherry for increasing GLP-1 levels for delaying the onset of obesity. In some embodiments, the formulation comprises effective amounts of L-arginine, resveratrol, and at least one extract from a tart cherry for increasing GLP-1 levels for reducing the likelihood of obesity. In some embodiments, the formulation comprises effective amounts of L-arginine, resveratrol, and at least one extract from a tart cherry for increasing GLP-1 levels for treating obesity. In some embodiments, the formulation comprises effective amounts of L-arginine, resveratrol, and at least one extract from a tart cherry for increasing GLP-1 levels for preventing obesity.

In some embodiments, the formulation comprises synergistically effective amounts of L-arginine, resveratrol, and at least one extract from a tart cherry for increasing GLP-1 levels for inhibiting, ameliorating, delaying the onset of, reducing the likelihood of, treating, and/or preventing obesity. In some embodiments, the formulation comprises synergistically effective amounts of L-arginine, resveratrol, and at least one extract from a tart cherry for increasing GLP-1 levels for inhibiting obesity. In some embodiments, the formulation comprises synergistically effective amounts of L-arginine, resveratrol, and at least one extract from a tart cherry for increasing GLP-1 levels for ameliorating obesity. In some embodiments, the formulation comprises synergistically effective amounts of L-arginine, resveratrol, and at least one extract from a tart cherry for increasing GLP-1 levels for delaying the onset of obesity. In some embodiments, the formulation comprises synergistically effective amounts of L-arginine, resveratrol, and at least one extract from a tart cherry for increasing GLP-1 levels for reducing the likelihood of obesity. In some embodiments, the formulation comprises synergistically effective amounts of L-arginine, resveratrol, and at least one extract from a tart cherry for increasing GLP-1 levels for treating obesity. In some embodiments, the formulation comprises synergistically effective amounts of L-arginine, resveratrol, and at least one extract from a tart cherry for increasing GLP-1 levels for preventing obesity.

In some embodiments, the formulation comprises effective amounts of L-arginine, resveratrol, and at least one extract from a tart cherry for increasing GLP-1 levels for inhibiting, ameliorating, delaying the onset of, reducing the likelihood of, treating, and/or preventing diabetes. In some embodiments, the formulation comprises effective amounts of L-arginine, resveratrol, and at least one extract from a tart cherry for increasing GLP-1 levels for inhibiting diabetes. In some embodiments, the formulation comprises effective amounts of L-arginine, resveratrol, and at least one extract from a tart cherry for increasing GLP-1 levels for ameliorating diabetes. In some embodiments, the formulation comprises effective amounts of L-arginine, resveratrol, and at least one extract from a tart cherry for increasing GLP-1 levels for delaying the onset of diabetes. In some embodiments, the formulation comprises effective amounts of L-arginine, resveratrol, and at least one extract from a tart cherry for increasing GLP-1 levels for reducing the likelihood of diabetes. In some embodiments, the formulation comprises effective amounts of L-arginine, resveratrol, and at least one extract from a tart cherry for increasing GLP-1 levels for treating diabetes. In some embodiments, the formulation comprises effective amounts of L-arginine, resveratrol, and at least one extract from a tart cherry for increasing GLP-1 levels for preventing diabetes.

In some embodiments, the formulation comprises synergistically effective amounts of L-arginine, resveratrol, and at least one extract from a tart cherry for increasing GLP-1 levels for inhibiting, ameliorating, delaying the onset of, reducing the likelihood of, treating, and/or preventing diabetes. In some embodiments, the formulation comprises synergistically effective amounts of L-arginine, resveratrol, and at least one extract from a tart cherry for increasing GLP-1 levels for inhibiting diabetes. In some embodiments, the formulation comprises synergistically effective amounts of L-arginine, resveratrol, and at least one extract from a tart cherry for increasing GLP-1 levels for ameliorating diabetes. In some embodiments, the formulation comprises synergistically effective amounts of L-arginine, resveratrol, and at least one extract from a tart cherry for increasing GLP-1 levels for delaying the onset of diabetes. In some embodiments, the formulation comprises synergistically effective amounts of L-arginine, resveratrol, and at least one extract from a tart cherry for increasing GLP-1 levels for reducing the likelihood of diabetes. In some embodiments, the formulation comprises synergistically effective amounts of L-arginine, resveratrol, and at least one extract from a tart cherry for increasing GLP-1 levels for treating diabetes. In some embodiments, the formulation comprises synergistically effective amounts of L-arginine, resveratrol, and at least one extract from a tart cherry for increasing GLP-1 levels for preventing diabetes.

### Formulations and Methods for Increasing GLP-1 Levels

In some embodiments, the present disclosure is related to formulations for increasing GLP-1 levels for inhibiting, ameliorating, delaying the onset of, reducing the likelihood of, treating, and/or preventing obesity and diabetes. An embodiment of the formulations according to the present disclosure is referred to as GLP-1^{Rx}. An embodiment of GLP-1^{Rx} is provided in the form of a dietary powder supplement. An embodiment of GLP-1^{Rx} is provided in the form of a dietary powder supplement for managing/maintaining/regulating blood sugar levels. An embodiment of GLP-1^{Rx} is provided in the form of a dietary powder supplement for promoting/enhancing weight loss. The ingredients of an embodiment of GLP-1^{Rx} is shown in Table 3:

TABLE 3
SUPPLEMENT FACTS
Servig Size: 7.75 g/1 scoop
Service per container 60

| | Amount | | |
|---|---|---|---|
| | per | | % Daily |
| | Serving | | Value |
| Calories | 35 | Cal | |
| Protein | 10 | g | 20%* |
| Vitamin C (as ascorbic acid) | 50 | mg | 56% |
| L-Arginine | 4500 | g | ** |
| Cherry Tart Powder | 250 | mg | ** |
| Reservatrol (from Polygonum | 100 | mg | ** |

cuspidatum [root] Extract

*Percent Daily Values are based on a 2,000 calorie diet.
** Daily Value (DV) not established.
Other ingredients: Citric Acid, Malic Acid, Natural Flavors, Stevia Leaf Extract, Beta-Carotene

In some embodiments, an amount of at least one extract from a tart cherry ranges from about 50 mg to about 1000 mg. In some embodiments, an amount of at least one extract from a tart cherry ranges from about 250 mg to about 2500 mg. In some embodiments, an amount of at least one extract from a tart cherry ranges from about 100 mg to about 400 mg. In some embodiments, an amount of at least one extract from a tart cherry is about 25, 50, 75, 100, 150, 200, 250, 300, 350, 400, 450, 500, 600, 700, 800, 900, 1000, or 2500, or a value within a range defined by any two of the aforementioned values.

In some embodiments, an effective amount of at least one extract from a tart cherry ranges from 50 mg to 1000 mg. In some embodiments, an effective amount of at least one extract from a tart cherry ranges from 250 mg to 2500 mg. In some embodiments, an effective amount of at least one extract from a tart cherry ranges from 100 mg to 400 mg. In some embodiments, an effective amount of at least one extract from a tart cherry is 25, 50, 75, 100, 150, 200, 250, 300, 350, 400, 450, 500, 600, 700, 800, 900, 1000, or 2500, or a value within a range defined by any two of the aforementioned values.

In some embodiments, an amount of at least one extract from a tart cherry ranges from about 5 g to about 10 g. In some embodiments, an amount is provided in 5 to 10 servings. In some embodiments, an amount is provided in 10 to 15 servings. In some embodiments, an amount is provided in 15 to 20 servings. In some embodiments, an amount is provided in 20 to 25 servings. In some embodiments, an amount is provided in 25 to 30 servings. In some embodiments, an amount is provided in 30 to 35 servings. In some embodiments, an effective amount is provided in 35 to 40 servings. In some embodiments, an amount is provided in 40 to 45 servings. In some embodiments, an amount is provided in 45 to 50 servings. In some embodiments, an amount is provided in 50 to 55 servings. In some embodiments, an amount is provided in 55 to 60 servings. In some embodiments, an amount is provided in 5, 10, 15, 20, 25, 30, 30, 35, 40, 45, 50, or 60 servings, or a value within a range defined by any two of the aforementioned values.

In some embodiments, an effective amount of at least one extract from a tart cherry ranges from 5 g to 10 g. In some embodiments, an effective amount is provided in 5 to 10 servings. In some embodiments, an effective amount is provided in 10 to 15 servings. In some embodiments, an effective amount is provided in 15 to 20 servings. In some embodiments, an effective amount is provided in 20 to 25 servings. In some embodiments, an effective amount is provided in 25 to 30 servings. In some embodiments, an effective amount is provided in 30 to 35 servings. In some embodiments, an effective amount is provided in 35 to 40 servings. In some embodiments, an effective amount is provided in 40 to 45 servings. In some embodiments, an effective amount is provided in 45 to 50 servings. In some embodiments, an effective amount is provided in 50 to 55 servings. In some embodiments, an effective amount is provided in 55 to 60 servings. In some embodiments, an effective amount is provided in 5, 10, 15, 20, 25, 30, 30, 35, 40, 45, 50, or 60 servings, or a value within a range defined by any two of the aforementioned values.

In some embodiments, an amount of resveratrol ranges from about 2 g to about 4 g. In some embodiments, an amount is provided in 5 to 10 servings. In some embodiments, an amount is provided in 10 to 15 servings. In some embodiments, an amount is provided in 15 to 20 servings. In some embodiments, an amount is provided in 20 to 25 servings. In some embodiments, an effective amount is provided in 25 to 30 servings. In some embodiments, an amount is provided in 30 to 35 servings. In some embodiments, an amount is provided in 35 to 40 servings. In some embodiments, an amount is provided in 40 to 45 servings. In some embodiments, an amount is provided in 45 to 50 servings. In some embodiments, an amount is provided in 50 to 55 servings. In some embodiments, an amount is provided in 55 to 60 servings. In some embodiments, an amount is provided in 5, 10, 15, 20, 25, 30, 30, 35, 40, 45, 50, or 60 servings, or a value within a range defined by any two of the aforementioned values.

In some embodiments, an effective amount of resveratrol ranges from 2 g to 4 g. In some embodiments, an effective amount is provided in 5 to 10 servings. In some embodiments, an effective amount is provided in 10 to 15 servings. In some embodiments, an effective amount is provided in 15 to 20 servings. In some embodiments, an effective amount is provided in 20 to 25 servings. In some embodiments, an effective amount is provided in 25 to 30 servings. In some embodiments, an effective amount is provided in 30 to 35 servings. In some embodiments, an effective amount is provided in 35 to 40 servings. In some embodiments, an effective amount is provided in 40 to 45 servings. In some embodiments, an effective amount is provided in 45 to 50 servings. In some embodiments, an effective amount is provided in 50 to 55 servings. In some embodiments, an effective amount is provided in 55 to 60 servings. In some embodiments, an effective amount is provided in 5, 10, 15, 20, 25, 30, 30, 35, 40, 45, 50, or 60 servings, or a value within a range defined by any two of the aforementioned values.

In some embodiments, an amount of resveratrol ranges from about 40 mg/day to about 750 mg/day. In some embodiments, an amount of resveratrol ranges from about 200 mg/day to about 1500 mg/day. In some embodiments, an amount of resveratrol is about 200 mg/day. In some embodiments, an amount of resveratrol is about 300 mg/day. In some embodiments, an amount of resveratrol is about 40, 80, 120, 160, 200, 250, 300, 350, 400, 450, 500, 550, 600, 650, 700, 750, 800, 850, 900, 950, 1000, 1250, or 1500 mg/day, or a value within a range defined by any two of the aforementioned values.

In some embodiments, an effective amount of resveratrol ranges from 40 mg/day to 750 mg/day. In some embodiments, an effective amount of resveratrol ranges from 200 mg/day to 1500 mg/day. In some embodiments, an effective amount of resveratrol is 200 mg/day. In some embodiments, an effective amount of resveratrol is 300 mg/day. In some embodiments, an effective amount of resveratrol is 40, 80, 120, 160, 200, 250, 300, 350, 400, 450, 500, 550, 600, 650, 700, 750, 800, 850, 900, 950, 1000, 1250, or 1500 mg/day, or a value within a range defined by any two of the aforementioned values.

In some embodiments, an amount of L-arginine ranges from about 90 g to about 180 g. In some embodiments, an amount is provided in 5 to 10 servings. In some embodiments, an amount is provided in 10 to 15 servings. In some embodiments, an amount is provided in 15 to 20 servings. In some embodiments, an amount is provided in 20 to 25 servings. In some embodiments, an amount is provided in 25 to 30 servings. In some embodiments, an amount is provided in 30 to 35 servings. In some embodiments, an amount is provided in 35 to 40 servings. In some embodiments, an amount is provided in 40 to 45 servings. In some embodiments, an amount is provided in 45 to 50 servings. In some embodiments, an amount is provided in 50 to 55 servings. In some embodiments, an effective amount is provided in 55 to 60 servings. In some embodiments, an amount is provided in 5, 10, 15, 20, 25, 30, 30, 35, 40, 45, 50, or 60 servings, or a value within a range defined by any two of the aforementioned values.

In some embodiments, an effective amount of L-arginine ranges from 90 g to 180 g. In some embodiments, an effective amount is provided in 5 to 10 servings. In some embodiments, an effective amount is provided in 10 to 15 servings. In some embodiments, an effective amount is provided in 15 to 20 servings. In some embodiments, an effective amount is provided in 20 to 25 servings. In some embodiments, an effective amount is provided in 25 to 30 servings. In some embodiments, an effective amount is provided in 30 to 35 servings. In some embodiments, an effective amount is provided in 35 to 40 servings. In some embodiments, an effective amount is provided in 40 to 45 servings. In some embodiments, an effective amount is provided in 45 to 50 servings. In some embodiments, an effective amount is provided in 50 to 55 servings. In some embodiments, an effective amount is provided in 55 to 60 servings. In some embodiments, an effective amount is provided in 5, 10, 15, 20, 25, 30, 30, 35, 40, 45, 50, or 60 servings, or a value within a range defined by any two of the aforementioned values.

In some embodiments, an amount of L-arginine ranges from about 1.5 g/day to about 30 g/day. In some embodiments, an amount of L-arginine ranges from about 9 g/day to about 45 g/day. In some embodiments, an amount of L-arginine is about 9 gm/day. In some embodiments, an amount of L-arginine is about 1.5, 3, 4.5, 6, 7.5, 9, 10, 12.5 15, 17.5 20, 22.5, 25, 27.5, 30, 32.5, 35, 37.5, 40, 42.5, or 45 g/day, or a value within a range defined by any two of the aforementioned values.

In some embodiments, an effective amount of L-arginine ranges from 1.5 g/day to 30 g/day. In some embodiments, an effective amount of L-arginine ranges from 9 g/day to 45 g/day. In some embodiments, an effective amount of L-arginine is 9 gm/day. In some embodiments, an effective amount of L-arginine is 1.5, 3, 4.5, 6, 7.5, 9, 10, 12.5 15, 17.5 20, 22.5, 25, 27.5, 30, 32.5, 35, 37.5, 40, 42.5, or 45 g/day, or a value within a range defined by any two of the aforementioned values.

In some embodiments, additional components in the formulation include at least one antioxidant vitamin and/or at least one prebiotic.

In some embodiments, additional components in the formulation include at least one antioxidant vitamin is Vitamin C.

In some embodiments, additional components in the formulation include at least one prebiotic is inulin.

TABLE 0.3
Embodiments of formulations with synergistically effective amounts of
L-arginine, resveratrol, and/or at least one extract from a tart cherry

| | Formulation | Formulation | Formulation | Formulation |
|---|---|---|---|---|
| Ingredients | 1 | 2 | 3 | 4 |
| At least one | + | + | + | |
| extract from | | | | |
| a tart cherry | | | | |
| Resveratrol | + | | + | + |
| L-arginine | + | + | | + |

TABLE 0.4
Embodiments of formulations with synergistically effective doses of
L-arginine, resveratrol, and/or at least one extract from a tart cherry

| | Formulation | Formulation | Formulation | Formulation |
|---|---|---|---|---|
| Ingredients | 1 | 2 | 3 | 4 |
| At least one | + | + | + | |
| extract from | | | | |
| a tart cherry | | | | |
| Resveratrol | + | | + | + |
| L-arginine | + | + | | + |

In some embodiments, the formulations comprise at least one extract from a tart cherry from Montmorency cherries, Balaton cherries, or a mixture of Montmorency and Balaton cherries.

In some embodiments, resveratrol is obtained from an extract of grape seed, grape seed oil, or grape skin.

The formulations additionally and/or optionally comprise one or more antioxidant vitamins. In some embodiments, the one or more antioxidant vitamins includes beta-carotene, vitamin C, and vitamin E.

The formulations additionally and/or optionally comprise one or more prebiotics. In some embodiments, the one or more prebiotics include inulin. Non-limiting examples of sources of prebiotic can include chicory root, garlic, dandelion greens, Jerusalem artichoke, onions, leeks, asparagus, bananas, barley, oats, apples, Konjac root, cocoa, Burdock root, flax seeds, Yacon root, jicama root, wheat bran, and seaweed.

Disclosed herein are embodiments of formulations comprising various combinations of at least one extract from a tart cherry, resveratrol, and L-arginine are for inhibiting, ameliorating, delaying the onset of, reducing the likelihood of, treating, and/or preventing obesity. In some embodiments, the formulations comprising various combinations of at least one extract from a tart cherry, resveratrol, and L-arginine are for inhibiting, ameliorating, delaying the onset of, reducing the likelihood of, treating, and/or preventing diabetes.

In some embodiments, the formulation is an oral supplement. In some embodiments, the formulation is an edible solid or an edible semi-solid. In some embodiments, the formulation is a potable liquid. In some embodiments, the formulation is a powder or pellet that can be reconstituted in a potable liquid (e.g., water). In some embodiments, the formulation is a tablet or a capsule that can be taken without reconstitution.

Embodiments of the formulations provided herein comprise active ingredients, inactive ingredients, excipients, additives, and/or pharmaceutically acceptable carriers. Examples of additives include natural polymer compounds, inorganic salts, binders, lubricants, disintegrants, surfactants, thickeners, coating agents, pH adjusters, antioxidants, flavoring agents, preservatives, and colorants among others. Examples of other pharmaceutically acceptable carriers include liquid carriers such as water, alcohol, emulsion, and solid carriers such as gel, powder, etc. Standard pharmaceutical formulation techniques and ingredients can be used, such as those disclosed in Remington's The Science and Practice of Pharmacy, 21st Ed., Lippincott Williams & Wilkins (2005), which is hereby incorporated by reference in its entirety.

Embodiments of the formulations for oral administration can be any dosage form that is suitable for oral ingestion, for example, liquid formulations such as elixir, suspension, syrup, emulsion, ampoule, a premixed ready-to-consume drink, etc., solid formulations such as gel, gum, drop, powder, granule, pill, sugar-coated tablet, film-coated tablet, capsule, package agent, etc. Also contemplated are sustained-release formulations such as gel-coated formulations, multi-coated formulations, localized release formulations.

In some embodiments, the formulation is provided in the form of a tablet, capsule, gel cap, or softgel. The tablet, capsule, gel cap, or softgel may comprise additional inactive ingredients such as fillers including, but not limited to, rice flour, methylcellulose, magnesium stearate. Several tablets, capsules, gel caps, or softgels typically comprise one daily dose of each of the active ingredients, as described below.

In some embodiments, the formulation is a single serving formulation to be taken once per day by a subject. Thus, in some embodiments a daily dose of active ingredients is provided in a single serving. In some embodiments one daily dose may be divided among two or more servings to be taken in a day, for example among two or more servings of a liquid beverage or two or more tablets. In some embodiments, a daily dose of the formulation can be taken by or administered to a subject in a 24-hour period. In some embodiments, one-half of a daily dose of the formulation can be taken by or administered to a subject in a 12-hour period. In some embodiments, one-third of a daily dose of the formulation can be taken by or administered to a subject in a 8-hour period. In some embodiments, one-fourth of a daily dose of the formulation can be taken by or administered to a subject in a 6-hour period. In some embodiments, one-sixth of a daily dose of the formulation can be taken by or administered to a subject in a 4-hour period. In some embodiments, a daily dose of the formulation is taken every day for 1 week to 12 months. In some embodiments, the formulation is taken for 1, 2, 3 or 4 weeks. In some embodiments, the formulation is taken for 2, 3, 4, 5, 6, 7, 8, 9, 10, 11 or 12 months, or a value within a range defined by any two of the aforementioned values.

In some embodiments, a daily dose of the formulation can be provided in one or more daily servings. In some embodiments, a daily dose is provided in a single serving. In some embodiments, a daily dose may be provided in two servings (up to 2 servings in 24 hours). In some embodiments, a daily dose may be provided in three servings (up to 3 servings in 24 hours). In some embodiments, a daily dose may be provided in four servings (up to 4 servings in 24 hours). In some embodiments, a daily dose may be provided in two or more servings (up to 5 servings in 24 hours). In some embodiments, a daily dose may be provided in six servings (up to 6 servings in 24 hours). While the size of each serving may vary, the quantity of active ingredients to be consumed will be equal to one serving. In the case of a single serving, where one single serving is intended to be taken per day, the single serving will comprise a complete daily dose. In the case of multiple servings, the quantity of active ingredients will be such that the total amount of active ingredients in the servings to be taken in a single day is equivalent to a daily dose.

In some embodiments, the formulation is provided in the form of a liquid drink. The liquid drink may comprise the active ingredients in a water base, or in another base liquid that is inert with respect to the active ingredients. The liquid drink may comprise one or more additional inactive ingredients. In some embodiment the liquid drink may comprise one or more gelling or stabilizing agents including, but not limited, to xanthan gum, guar gum, propylene glycol, acacia gum and maltodextrin. In some embodiments the liquid drink may comprise one or more colorants, for example a natural color such as carmine or beet root. In some embodiments the liquid drink may comprise one or more preservatives such as citrus oil, sorbates and benzoates. The liquid drink may also comprise one or more flavors such as fruit (e.g., strawberry, melon, apple, peach, lemon, orange, mango, and the like) and non-fruit flavors. In some embodiments the flavor is a natural flavor. In some embodiments the liquid drink may comprise artificial and natural sweeteners.

In some embodiments the formulation comprises, but is not limited to, one or more artificial sweeteners such as sucralose, acesulfame K or a combination of these and/or one or more natural sweeteners such as sugar, fructose, honey, erythritol, xylitol, stevia, monk fruit, agave, citrus and protein extracts used as sweeteners or combinations thereof.

In some embodiments, the formulation is a liquid drink comprising a single serving of about 4.5 fl. oz. However, the skilled artisan will appreciate that while the size of the serving itself may vary depending on the quantity of the inactive ingredients, such as water, the amount of the active ingredients in each single serving will be within the ranges provided herein. For example, while the single serving size may be ½ fl. oz, 1 fl. oz, 2 fl. oz., 4 fl. oz., 6 fl. oz., 10 fl. oz., 12 fl. oz., 16 fl. oz., or a value within a range defined by any two of the aforementioned values, or anything smaller or larger, the daily dose of active ingredients will be the same in each serving. In some embodiments a single serving is about 2 fl. oz. to about 54 fl. oz. In some embodiments, the formulation is a liquid concentrate comprising of multiple servings to be diluted into a liquid beverage to obtain a single serving size.

In some embodiments, the formulation is a dietary powder supplement comprising a single serving of 7.75 g (1 scoop) (Table 3). In some embodiments, the formulation is a dietary powder supplement comprising a single serving of 1 g-2.5 g. In some embodiments, the formulation is a dietary powder supplement comprising a single serving of 2.5 g-5 g. In some embodiments, the formulation is a dietary powder supplement comprising a single serving of 5 g-7.5 g. In some embodiments, the formulation is a dietary powder supplement comprising a single serving of 7.5 g-10 g. In some embodiments, the formulation is a dietary powder supplement comprising a single serving of 10 g-12.5 g. In some embodiments, the formulation is a dietary powder supplement comprising a single serving of 12.5 g-15 g. In some embodiments, the formulation is a dietary powder supplement comprising a single serving of about 1 g-about 2.5 g. In some embodiments, the formulation is a dietary powder supplement comprising a single serving of about 2.5 g-about 5 g. In some embodiments, the formulation is a dietary powder supplement comprising a single serving of about 5 g-about 7.5 g. In some embodiments, the formulation is a dietary powder supplement comprising a single serving of about 7.5 g-about 10 g. In some embodiments, the formulation is a dietary powder supplement comprising a single serving of about 10 g-about 12.5 g. In some embodiments, the formulation is a dietary powder supplement comprising a single serving of about 12.5 g-about 15 g. In some embodiments, the formulation is a dietary powder supplement comprising a single serving of 1, 2, 3, 4, 5, 6, 7, 8, 9, 10, 11, 12, 13, 14, or 15 g, or a value within a range defined by any two of the aforementioned values. In some embodiments, the formulation is a dietary powder supplement comprising a single serving of about 1, 2, 3, 4, 5, 6, 7, 8, 9, 10, 11, 12, 13, 14, or 15 g, or a value within a range defined by any two of the aforementioned values.

In some embodiments, the formulation comprises one or more additional components that improve absorption of one or more of the active ingredients.

In some embodiments, the formulation is taken in the morning on an empty stomach. In some embodiments, the formulation is taken at any time enhanced energy levels are needed. In some embodiments, the formulation is taken daily to provide enhanced energy levels. In some embodiments, the formulation is taken to promote a feeling of overall well-being.

In some embodiments, general health improvements after taking the formulation may include one or more of inhibition, amelioration, delayed onset of, reduced likelihood of, treatment, and/or prevention of metabolic syndrome, and increase in energy levels. In some embodiments, general health improvements after taking the formulation includes include one or more of inhibition, amelioration, delayed onset of, reduced likelihood of, treatment, and/or prevention of metabolic syndrome. In some embodiments, general health improvements after taking the formulation includes increase in energy levels. In some embodiments, general health improvements after taking the formulation includes inhibition, amelioration, delayed onset of, reduced likelihood of, treatment, and prevention of metabolic syndrome, as well as increase in energy levels.

In some embodiments, general health improvements including, but not limited to, inhibition, amelioration, delayed onset of, reduced likelihood of, treatment, and/or prevention of metabolic syndrome, and/or increase in energy levels are observed within 1 day to about 21 days of taking an embodiment of the formulations herein. In some embodiments, general health improvements are observed within 1 day to about 7 days. In some embodiments, general health improvements are observed within 1 day to about 14 days. In some embodiments, general health improvements are observed within 1 day to about 5 days. In some embodiments, general health improvements are observed within 1 day to about 10 days. In some embodiments, general health improvements are observed within 1 day to about 15 days. In some embodiments, general health improvements are observed within 1 day to about 20 days.

In some embodiments, inhibition, amelioration, delayed onset of, reduced likelihood of, treatment, and/or prevention of obesity and diabetes may also be associated with high levels of concentration, optimal cognitive function, effective decision making, keen comprehension, effortless recall, mental clarity, quick reflexes, restful sleep, increased motivation, coordinated motor function, efficient problem solving, mental acuity, optimal energy levels, overall well-being, ability to focus, mood stability, memory support, alertness, improved neurotransmission, and nerve cell integrity.

In some embodiments, the formulations may provide benefits to subjects suffering from other conditions related to metabolic syndrome and lower energy levels. Non-limiting examples include Post Traumatic Stress Disorder (PTSD), Attention Deficit Disorder (ADD), Attention Deficit Hyperactivity Disorder (ADHD), anxiety, migraine, dementia, tinnitus, and in hangovers.

In some embodiments, formulations comprising combinations of at least one extract from a tart cherry, resveratrol, and L-arginine are provided. In some embodiments, formulations comprising sub-combinations of at least one extract from a tart cherry, resveratrol, and L-arginine are provided. In some embodiments, the combinations and sub-combinations further comprise one or more antioxidant vitamin. In some embodiments, the combinations and sub-combinations further comprise Vitamin C. In some embodiments, the combinations and sub-combinations further comprise one or more prebiotics. In some embodiments, the combinations and sub-combinations further comprise inulin.

In some embodiments, the combinations and sub-combinations are intended to target the biochemical pathways to increase GLP-1 levels. For example, L-arginine increases secretion of natural GLP-1, resulting in an increase GLP-1 levels. Resveratrol inhibits DPP4 thereby preventing degradation of GLP-1 by DPP-4 thereby prolonging the half-life of GLP-1 and further increasing GLP-1 levels. Tart cherry extract contains multiple components that inhibit arginase, which maintains/enhances levels of L-arginine, which in turn increases natural secretion of GLP-1, thereby further increasing GLP-1 levels. Overall, the combination of L-arginine, resveratrol, and tart cherry act synergistically to increase GLP-1 levels.

In some embodiments, the ingredients in the combinations and sub-combinations act synergistically to amplify the effect of formulation in inhibiting, ameliorating, delaying the onset of, reducing the likelihood of, treating, and/or preventing obesity and diabetes. For example, without being limited by any particular theory, L-arginine increases secretion of natural GLP-1, resulting in an increase GLP-1 levels. Resveratrol inhibits DPP4 thereby preventing degradation of GLP-1 by DPP-4 thereby prolonging the half-life of GLP-1 and further increasing GLP-1 levels. Tart cherry extract contains multiple components that inhibit arginase, which maintains/enhances levels of L-arginine, which in turn increases natural secretion of GLP-1, thereby further increasing GLP-1 levels. Overall, the combination of L-arginine, resveratrol, and tart cherry act synergistically to increase GLP-1 levels thereby inhibiting, ameliorating, delaying the onset of, reducing the likelihood of, treating, and/or preventing obesity and diabetes.

In some embodiments, the formulation comprises at least 3 active ingredients that are synergistic in their effects. In some embodiments, the formulation comprises at least 3, 4, 5, 6, 7, 8, 9, 10, 11, 12 or 13 synergistic active ingredients, or a value within a range defined by any two of the aforementioned values. For example, the active ingredients may be synergistic with respect to their positive effects with respect to inhibiting, ameliorating, delaying the onset of, reducing the likelihood of, treating, and/or preventing diabetes and/or obesity. In some embodiments, the active ingredients may be synergistic with respect to their positive effects with respect to inhibiting, ameliorating, delaying the onset of, reducing the likelihood of, treating, and/or preventing diabetes. In some embodiments, the active ingredients may be synergistic with respect to their positive effects with respect to inhibiting, ameliorating, delaying the onset of, reducing the likelihood of, treating, and/or preventing obesity.

In some embodiments, the effects of 3 or more of the active ingredients are additive. In some embodiments, the effects of at least 3, 4, 5, 6, 7, 8, 9, 10, 11, 12 or 13 of the active ingredients are additive, or a value within a range defined by any two of the aforementioned values. For example, the effects may be additive with respect to having positive effects with respect to inhibiting, ameliorating, delaying the onset of, reducing the likelihood of, treating, and/or preventing diabetes and/or obesity. In some embodiments, the active ingredients may be additive with respect to their positive effects with respect to inhibiting, ameliorating, delaying the onset of, reducing the likelihood of, treating, and/or preventing diabetes. In some embodiments, the active ingredients may be additive with respect to their positive effects with respect to inhibiting, ameliorating, delaying the onset of, reducing the likelihood of, treating, and/or preventing obesity.

In some embodiments, the effects of the at least 3-13 active ingredients are additive. In some embodiments, the effects of the at least 3-13 active ingredients are synergistic. In some embodiments, the effects of the at least 3-13 active ingredients are additive and/or synergistic.

In some embodiments, the formulations comprise about 1500 mg of at least one extract from a tart cherry. In some embodiments, the formulations comprise about 250 mg to about 2500 mg of at least one extract from a tart cherry. In some embodiments, the formulations comprise about 500 mg to about 5000 mg of at least one extract from a tart cherry. In some embodiments, the formulations comprise about 750 mg to about 7500 mg of at least one extract from a tart cherry. In some embodiments, the formulations comprise about 1000 mg to about 15000 mg of at least one extract from a tart cherry.

In some embodiments, the formulations comprise about 250 mg of Vitamin C. In some embodiments, the formulations comprise about 25 mg to about 500 mg of Vitamin C. In some embodiments, the formulations comprise about 100 mg to about 1000 mg of Vitamin C. In some embodiments, the formulations comprise about 200 mg to about 2000 mg of Vitamin C. In some embodiments, the formulations comprise about 500 mg to about 2500 mg of Vitamin C.

In some embodiments, the formulations comprise about 500 mg of Inulin. In some embodiments, the formulations comprise about 50 mg to about 350 mg of Inulin. In some embodiments, the formulations comprise about 300 mg to about 1500 mg of Inulin. In some embodiments, the formulations comprise about 2000 mg to about 3500 mg of Inulin. In some embodiments, the formulations comprise about 3500 mg to about 5000 mg of Inulin.

In some embodiments, the formulation comprises the active ingredients Table 0.5 in the indicated recommended amounts in one dose or serving of the formulation, for example in a daily dose:

TABLE 0.5

| Active ingredient | Amounts in one dose or serving |
|---|---|
| At least one | about 0.25 gm/day to about 5 gm/day. |
| extract from a | Without being limited by any particular |
| tart cherry | theory, this dosage is equivalent to about |
| | 180 Montmorency cherries. |
| Resveratrol | about 40 mg/day to about 750 mg/day |
| L-arginine | about 1.5 g/day to about 30 g/day |
| Vitamin C | 0 gm/day to about 2 gm/day |
| Inulin | 0 gm/day to about 10 gm/day |

In some embodiments, the formulation comprises the active ingredients Table 0.6 in the indicated recommended amounts in one dose or serving of the formulation, for example in a daily dose:

TABLE 0.6

| Active ingredient | Amounts in one dose or serving |
|---|---|
| At least one | 0.25 gm/day to 5 gm/day. Without being |
| extract from a tart | limited by any particular theory, this dosage |
| cherry | is equivalent to about 180 Montmorency |
| | cherries. |
| Resveratrol | 40 mg/day to 750 mg/day |
| L-arginine | 1.5 g/day to 30 g/day |
| Vitamin C | 0 gm/day to 2 gm/day |
| Inulin | 0 gm/day to 10 gm/day |

In some embodiments, the methods result in formulations that comprise at least one extract from a tart cherry at a dose range of about 0.25 gm to about 5 gm per daily dose or serving.

In some embodiments, the methods result in formulations that comprise resveratrol at a dose range of about 40 mg/day to about 750 mg/day.

In some embodiments, the methods result in formulations that comprise L-arginine at a dose range of about 1.5 g/day to about 30 g/day.

In some embodiments, the formulations comprise Vitamin C at a dose range of about 0 gm to about 2 gm per daily dose or serving.

In some embodiments, the formulations comprise Inulin at a dose range of about 0 gm to about 10 gm per daily dose or serving.

In some embodiments of the formulation, an effective amount of the at least one extract from a tart cherry ranges from about 1.5 grams to about 2.5 grams. In some embodiments of the formulation, an effective amount of the at least one extract from a tart cherry ranges from about 0.15 gram to about 25 grams.

In some embodiments of the formulation, an effective amount of the at least one antioxidant vitamin ranges from about 250 mg to about 500 mg. In some embodiments of the formulation, an effective amount of the at least one antioxidant vitamin ranges from about 25 mg to about 5000 mg.

In some embodiments of the formulation, an effective amount of the at least one prebiotic is about 450 mg. In some embodiments of the formulation, an effective amount of the at least one prebiotic ranges from about 45 mg to about 4500 mg.

In some embodiments, an effective amount of at least one extract from a tart cherry in the formulation ranges from about 0.15 gram to about 25 grams. In some embodiments, an effective amount of at least one extract from a tart cherry in the formulation ranges from about 1.5 grams to about 2.5 grams. In some embodiments, an effective amount of at least one extract from a tart cherry in the formulation is about 0.15, 0.3, 0.45, 0.6, 0.75, 1, 2.5, 5, 7.5, 10, 12.5, 15, 17.5, 20, 22.5, or 25 grams, or a value within a range defined by any two of the aforementioned values.

In some embodiments, an effective amount of resveratrol in the formulation ranges from about 20 mg to about 2000 mg. In some embodiments, an effective amount of resveratrol is about 200 mg.

In some embodiments, an effective amount of L-arginine in the formulation ranges from about 1.5 g to about 30 g. In some embodiments, an effective amount of L-arginine in the formulation is about 20 g.

In some embodiments, an effective amount of Vitamin C in the formulation ranges from about 25 mg to about 5000 mg. In some embodiments, an effective amount of Vitamin C in the formulation ranges from about 250 mg to about 500 mg.

In some embodiments, an effective amount of Inulin in the formulation ranges from about 45 mg to about 4500 mg. In some embodiments, an effective amount of Inulin in the formulation is about 450 mg.

Any one or more of the embodiments of the formulations and kits of formulations disclosed herein can be used in methods for inhibiting, ameliorating, delaying the onset of, reducing the likelihood of, treating, and/or preventing diabetes and/or obesity.

Any one or more of the embodiments of the formulations and kits of formulations disclosed herein can be used in methods for inhibiting, ameliorating, delaying the onset of, reducing the likelihood of, treating, and/or preventing diabetes.

Any one or more of the embodiments of the formulations and kits of formulations disclosed herein can be used in methods for inhibiting, ameliorating, delaying the onset of, reducing the likelihood of, treating, and/or preventing obesity.

### Additional Embodiments

In some embodiments, a method for increasing GLP-1 levels in a subject is provided.

In some embodiments, the method for increasing GLP-1 levels comprises administering to the subject a formulation comprising an effective amount of at least one extract from a tart cherry, an effective amount of resveratrol, an effective amount of L-arginine. The effective amount of each ingredient is sufficient in combination for increasing GLP-1 levels.

In some embodiments of the method for increasing GLP-1 levels, the effective amount of the at least one extract from a tart cherry ranges from 250 mg to 1000 mg.

In some embodiments of the method for increasing GLP-1 levels, the effective amount of the resveratrol ranges from 100 mg to 400 mg.

In some embodiments of the method for increasing GLP-1 levels, the effective amount of the L-arginine ranges from 1.5 g to 9 g.

In some embodiments of the method for increasing GLP-1 levels, the formulation further comprises an effective amount of at least one antioxidant vitamin.

In some embodiments of the method for increasing GLP-1 levels, the least one antioxidant vitamin is Vitamin C.

In some embodiments of the method for increasing GLP-1 levels, an amount of the Vitamin C ranges from 25 mg to 500 mg.

In some embodiments of the method for increasing GLP-1 levels, the formulation further comprises an effective amount of at least one prebiotic.

In some embodiments of the method for increasing GLP-1 levels, the at least one prebiotic is inulin.

In some embodiments of the method for increasing GLP-1 levels, the effective amount of inulin ranges from 45 mg to 4500 mg.

In some embodiments, the method for increasing GLP-1 levels inhibits ameliorates, delays an onset of, reduces a likelihood of, treats, and/or prevents diabetes in the subject.

In some embodiments, the method for increasing GLP-1 levels inhibits ameliorates, delays an onset of, reduces a likelihood of, treats, and/or prevents obesity in the subject.

In some embodiments, a method for increasing GLP-1 levels in a subject is provided.

In some embodiments, the method for increasing GLP-1 levels comprises administering to the subject a formulation comprising an amount of at least one extract from a tart cherry, an amount of resveratrol, an amount of L-arginine. The amounts of the at least one extract from a tart cherry, the resveratrol, and the L-arginine are, in combination, effective in increasing GLP-1 levels.

In some embodiments of the method for increasing GLP-1 levels, the amount of the at least one extract from a tart cherry ranges from 250 mg to 1000 mg.

In some embodiments of the method for increasing GLP-1 levels, the amount of the resveratrol ranges from 100 mg to 400 mg.

In some embodiments of the method for increasing GLP-1 levels, the amount of the L-arginine ranges from 1.5 g to 9 g.

In some embodiments of the method for increasing GLP-1 levels, the formulation further comprises an amount of at least one antioxidant vitamin.

In some embodiments of the method for increasing GLP-1 levels, the least one antioxidant vitamin is Vitamin C.

In some embodiments of the method for increasing GLP-1 levels, an amount of the Vitamin C ranges from 25 mg to 500 mg.

In some embodiments of the method for increasing GLP-1 levels, the formulation further comprises an amount of at least one prebiotic.

In some embodiments of the method for increasing GLP-1 levels, the at least one prebiotic is inulin.

In some embodiments of the method for increasing GLP-1 levels, the amount of inulin ranges from 45 mg to 4500 mg.

In some embodiments, the method for increasing GLP-1 levels inhibits ameliorates, delays an onset of, reduces a likelihood of, treats, and/or prevents diabetes in a subject.

In some embodiments, the method for increasing GLP-1 levels inhibits ameliorates, delays an onset of, reduces a likelihood of, treats, and/or prevents obesity in a subject.

In some embodiments, a formulation is provided.

In some embodiments, the formulation comprises an effective amount of at least one extract from a tart cherry, an effective amount of resveratrol, an effective amount of L-arginine, and a pharmaceutically acceptable carrier.

In some embodiments of the formulation, the effective amount of the at least one extract from a tart cherry ranges from 250 mg to 1000 mg.

In some embodiments of the formulation, the effective amount of the resveratrol ranges from 100 mg to 400 mg.

In some embodiments of the formulation, the effective amount of the L-arginine ranges from 1.5 g to 9 g.

In some embodiments of the formulation, the formulation further comprises an effective amount of at least one antioxidant vitamin.

In some embodiments of the formulation, the least one antioxidant vitamin is Vitamin C.

In some embodiments of the formulation, an effective amount of the Vitamin C ranges from 25 mg to 500 mg.

In some embodiments of the formulation, the formulation further comprises an amount of at least one prebiotic.

In some embodiments of the formulation, the at least one prebiotic is inulin.

In some embodiments of the formulation, the effective amount of inulin ranges from 45 mg to 4500 mg.

In some embodiments, a formulation is provided.

In some embodiments, the formulation comprises an amount of at least one extract from a tart cherry, an amount of resveratrol, an amount of L-arginine, and a pharmaceutically acceptable carrier.

In some embodiments of the formulation, the amount of the at least one extract from a tart cherry ranges from 250 mg to 1000 mg.

In some embodiments of the formulation, the amount of the resveratrol ranges from 100 mg to 400 mg.

In some embodiments of the formulation, the amount of the L-arginine ranges from 1.5 g to 9 g.

In some embodiments of the formulation, the formulation further comprises an amount of at least one antioxidant vitamin.

In some embodiments of the formulation, the least one antioxidant vitamin is Vitamin C.

In some embodiments of the formulation, an amount of the Vitamin C ranges from 25 mg to 500 mg.

In some embodiments of the formulation, the formulation further comprises an amount of at least one prebiotic.

In some embodiments of the formulation, the at least one prebiotic is inulin.

In some embodiments of the formulation, the amount of inulin ranges from 45 mg to 4500 mg.

### Methods of Making Formulations for Increasing GLP-1 Levels

Also disclosed herein are embodiments of methods of making any of the formulations for increasing GLP-1 levels disclosed herein.

In some embodiments, methods are described for making the embodiments of the formulations herein, wherein the formulations comprise one or more ingredients that are prone to floatation, sedimentation, and/or precipitation. In some embodiments, methods of making a liquid dietary supplement drink using a combination of synergistic ingredients that target long and short-term human health are described in which the formation of floaties (precipitate) or bottom-settlement (sediment) in the formulation is reduced or avoided entirely.

In some embodiments, the generation of a sediment and/or precipitate can be reduced or avoided by combining the ingredients in one or more particular sequences. Thus, in some embodiments special attention is given to the order in which each ingredient is added. In particular, in some embodiments the ingredients are added in an order which results in all of the ingredients going into solution, or staying in suspension.

In addition to avoiding precipitation and sedimentation, other indicators of stability of the formulation can be preserved during preparation of the formulation. These indicators of stability of the formulation may include, for example, flavor profile, texture and mouth-feel. Thus, in some embodiments, one or more indicator of stability, such as flavor profile, texture and mouth-feel of the formulation prepared according to the methods described herein are substantially as desired.

In some embodiments, the ingredients are freeze dried to create a powder that minimizes loss of potency before preparing a formulation.

In some embodiments, any of the ingredients in the formulations can be the first ingredient to be added to water or other liquid in preparing a liquid formulation. The remaining ingredients may be added in any order or combination. Flavor components, colorants and sweeteners, if any, are typically added last.

In some embodiments, at least one extract from a tart cherry is the first ingredient that is added to water or other liquid in preparing a liquid formulation. The remaining ingredients may be added in any order or combination.

In some embodiments, resveratrol is the first ingredient that is added to water or other liquid in preparing a liquid formulation. The remaining ingredients may be added in any order or combination.

In some embodiments, L-arginine is the first ingredient that is added to water or other liquid in preparing a liquid formulation. The remaining ingredients may be added in any order or combination.

In some embodiments, Vitamin C is the last ingredient that is added to water or other liquid in preparing a liquid formulation. The remaining ingredients may be added in any order or combination.

In some embodiments, inulin is the last ingredient that is added to water or other liquid in preparing a liquid formulation. The remaining ingredients may be added in any order or combination.

In some embodiments, the formulation is prepared in the form of a liquid drink. The liquid drink may comprise the active ingredients in a water base, or in another base liquid that is inert with respect to the active ingredients. The liquid drink may comprise one or more additional inactive ingredients. In some embodiments the liquid drink may comprise one or more preservatives such as citrus oil, sorbates and benzoates. The liquid drink may also comprise one or more flavors such as fruit (e.g., strawberry, melon, apple, peach, lemon, orange, mango, and the like) and non-fruit flavors. In some embodiments the flavor is a natural flavor.

In some embodiments, sweeteners, such as sucralose or acesulfame K or a combination thereof, are added. In some embodiments, a natural sweetener such as sugar, fructose, honey, agave, monk fruit, stevia, erythritol, xylitol, citrus and protein extracts or a combination thereof are added.

In some embodiments, a liquid formulation is prepared by combining the following water, tart cherry extract, vitamin, curcumin, grape seed extract, and inulin. In some embodiments, inosine is optionally added. In some embodiments, creatine is optionally added. In some embodiments, inosine and creatine are optionally added.

In some embodiments, the ingredients are mixed in an order that allows for optimum solubility throughout the liquid phase. In some embodiments, the ingredients are mixed in an order that does not interfere with the dispersion and solubility of other ingredients throughout the liquid phase and does not, for example, lead to clumps (precipitation) and/or fall-out (sedimentation) throughout the liquid. In some embodiments, the ingredients are mixed in a specific order that is empirically determined and that allows for optimum solubility throughout the liquid phase. In some embodiments, the ingredients are mixed in an order that is empirically determined and that does not interfere with the dispersion and solubility of other ingredients throughout the liquid phase and does not, for example, lead to clumps (precipitation) and/or fall-out (sedimentation) throughout the liquid.

In some embodiments, one or more additional ingredients are added to water one or more premixes, each premix comprising a fraction of the other ingredients. The premixes may be added in multiple steps or in a single step.

In some embodiments, all additional ingredients are added to water as a single premix comprising all the additional ingredients.

In some embodiments, the time required for dissolving and/or dispersing the ingredients in water at least about 10 to about 60 minutes. In some embodiments, the time required is about 10 to about 30 minutes. In some embodiments, the time required is about 20 to about 60 minutes.

In some embodiments, the time required for dissolving and/or dispersing the ingredients in water at least 10 to 60 minutes. In some embodiments, the time required is 10 to 30 minutes. In some embodiments, the time required is 20 to 60 minutes.

In some embodiments, additional flavoring components are added to the formulation. In some embodiments, any and all flavoring components, such as Natural flavors, and sweeteners are added next, either individually and sequentially any order, or simultaneously. Exemplary sweeteners include, but are not limited to natural sweeteners, such as Sugar, Fructose, Honey, Agave, Monk fruit, Stevia, Erythritol, Xylitol, Citrus and Protein extracts, and artificial sweeteners such as Sucralose, Acesulfame K, or a combination of these.

The ingredients and flavoring components may be dissolved in the solution, for example, by one or more of the following techniques: gentle stirring, gentle vortexing, gentle heating. In some embodiments, the time required for dissolving these components is at least about 5 to about 60 minutes. In some embodiments, the time required is about 5 to about 30 minutes. In some embodiments, the time required is about 30 to about 60 minutes.

In some embodiments colorant or colorants are added next and dissolved in the solution, for example, by one or more of the following techniques: gentle stirring, gentle vortexing, gentle heating. In some embodiments, the time required for dissolving these components is at least about 5 to 20 minutes. In some embodiments, the time required is about 30 minutes to about 50 minutes. In some embodiments, the time required for dissolving these components is at least 5 to 20 minutes. In some embodiments, the time required is 30 minutes to 50 minutes.

In some embodiments one or more salts are added next and dissolved in the solution, for example, by one or more of the following techniques: gentle stirring, gentle vortexing, gentle heating. In some embodiments, the time required for dissolving the one or more salts is at least about 5 to 20 minutes. In some embodiments, the time required is about 30 minutes to about 50 minutes. In some embodiments, the time required for dissolving the one or more salts is at least 5 to 20 minutes. In some embodiments, the time required is 30 minutes to 50 minutes.

In some embodiments, the pH of the finished product is acidic. In some embodiments, the pH of the finished product is about 3 to about 6.5. In some embodiments, the pH of the finished product is about 3.0, 3.5, 4.0, 4.5, 5.0, 5.5, 6.0, or 6.5, or a value within a range defined by any two of the aforementioned values. In some embodiments, the pH of the finished product is 3 to 6.5. In some embodiments, the pH of the finished product is 3.0, 3.5, 4.0, 4.5, 5.0, 5.5, 6.0, or 6.5, or a value within a range defined by any two of the aforementioned values.

In some embodiments, the formulation comprises at least three active ingredients that are synergistic in their effects. In some embodiments, the formulation comprises at least 4, 5, 6, 7, 8, 9, 10, 11, 12 or 13 synergistic active ingredients, or a value within a range defined by any two of the aforementioned values. For example, the active ingredients may be synergistic with respect to their positive effects with respect to inhibiting, ameliorating, delaying the onset of, reducing the likelihood of, treating, and/or preventing obesity and diabetes.

In some embodiments, the methods result in formulations that comprise about 1500 mg of Tart cherry. In some embodiments, the methods result in formulations that about 250 mg to about 2500 mg of Tart cherry. In some embodiments, the methods result in formulations that about 500 mg to about 5000 mg of Tart cherry. In some embodiments, the methods result in formulations that comprise about 750 mg to about 7500 mg of Tart cherry. In some embodiments, the formulations comprise about 1000 mg to about 15000 mg of Tart cherry.

In some embodiments, the methods result in formulations that comprise 1500 mg of Tart cherry. In some embodiments, the methods result in formulations that 250 mg to 2500 mg of Tart cherry. In some embodiments, the methods result in formulations that 500 mg to 5000 mg of Tart cherry. In some embodiments, the methods result in formulations that comprise 750 mg to 7500 mg of Tart cherry. In some embodiments, the formulations comprise 1000 mg to 15000 mg of Tart cherry.

In some embodiments, the methods result in formulations that comprise about 200 mg of resveratrol. In some embodiments, the methods result in formulations that about 100 mg to about 400 mg of resveratrol.

In some embodiments, the methods result in formulations that comprise 200 mg of resveratrol. In some embodiments, the methods result in formulations that 100 mg to 400 mg of resveratrol.

In some embodiments, the methods result in formulations that comprise about 9 g of L-arginine. In some embodiments, the methods result in formulations that about 1.5 g to about 9 g of L-arginine.

In some embodiments, the methods result in formulations that comprise 9 g of L-arginine. In some embodiments, the methods result in formulations that 1.5 g to 9 g of L-arginine.

In some embodiments, the methods result in formulations that comprise about 250 mg of Vitamin C. In some embodiments, the methods result in formulations that comprise about 25 mg to about 500 mg of Vitamin C. In some embodiments, the methods result in formulations that comprise about 100 mg to about 1000 mg of Vitamin C. In some embodiments, the methods result in formulations that comprise about 200 mg to about 2000 mg of Vitamin C. In some embodiments, the methods result in formulations that comprise about 500 mg to about 2500 mg of Vitamin C.

In some embodiments, the methods result in formulations that comprise 250 mg of Vitamin C. In some embodiments, the methods result in formulations that comprise 25 mg to 500 mg of Vitamin C. In some embodiments, the methods result in formulations that comprise 100 mg to 1000 mg of Vitamin C. In some embodiments, the methods result in formulations that comprise 200 mg to 2000 mg of Vitamin C. In some embodiments, the methods result in formulations that comprise 500 mg to 2500 mg of Vitamin C.

In some embodiments, the methods result in formulations that comprise about 500 mg of Inulin. In some embodiments, the methods result in formulations that comprise about 50 mg to about 350 mg of Inulin. In some embodiments, the methods result in formulations that comprise about 300 mg to about 1500 mg of Inulin. In some embodiments, the methods result in formulations that comprise about 2000 mg to about 3500 mg of Inulin. In some embodiments, the methods result in formulations that comprise about 3500 mg to about 5000 mg of Inulin.

In some embodiments, the methods result in formulations that comprise 500 mg of Inulin. In some embodiments, the methods result in formulations that comprise 50 mg to 350 mg of Inulin. In some embodiments, the methods result in formulations that comprise 300 mg to 1500 mg of Inulin. In some embodiments, the methods result in formulations that comprise 2000 mg to 3500 mg of Inulin. In some embodiments, the methods result in formulations that comprise 3500 mg to 5000 mg of Inulin.

In some embodiments, the methods result in a formulation that comprises the active ingredients as shown in Table 0.7 in the indicated recommended amounts in one dose or serving of the formulation, for example in a daily dose:

TABLE 0.7

| Active Ingredient | Amount in one dose or serving |
|---|---|
| Tart cherry extract | about 0.25 gm/day to about 5 gm /day. Without |
| | being limited by any particular theory, this dosage |
| | is equivalent to about 180 Montmorency cherries. |
| Resveratrol | about 40 mg/day to about 750 mg/day |
| L-arginine | about 1.5 g/day to about 30 g/day |
| Vitamin C | 0 gm/day to about 2 gm/day |
| Inulin | 0 gm/day to about 10 gm/day. |

In some embodiments, the methods result in a formulation that comprises the active ingredients as shown in Table 0.8 in the indicated recommended amounts in one dose or serving of the formulation, for example in a daily dose:

TABLE 0.8

| Active Ingredient | Amount in one dose or serving |
|---|---|
| Tart cherry extract | 0.25 gm/day to 5 gm/day. Without being limited by |
| | any particular theory, this dosage is equivalent to |
| | about 180 Montmorency cherries. |
| Resveratrol | 40 mg/day to 750 mg/day |
| L-arginine | 1.5 g/day to 30 g/day |
| Vitamin C | 0 gm/day to 2 gm/day |
| Inulin | 0 gm/day to 10 gm/day. |

In some embodiments, the methods result in formulations that comprise at least one extract from a tart cherry at a dose range of about 0.25 gm to about 5 gm per daily dose or serving. In some embodiments, the methods result in formulations that comprise at least one extract from a tart cherry at a dose range of 0.25 gm to 5 gm per daily dose or serving.

In some embodiments, the methods result in formulations that comprise resveratrol at a dose range of about 40 mg/day to about 750 mg/day. In some embodiments, the methods result in formulations that comprise resveratrol at a dose range of 40 mg/day to 750 mg/day.

In some embodiments, the methods result in formulations that comprise L-arginine at a dose range of about 1.5 g/day to about 30 g/day. In some embodiments, the methods result in formulations that comprise L-arginine at a dose range of 1.5 g/day to 30 g/day.

In some embodiments, the methods result in formulations that comprise Vitamin C at a dose range of about 0 gm to about 2 gm per daily dose or serving. In some embodiments, the methods result in formulations that comprise Vitamin C at a dose range of about 0 gm to 2 gm per daily dose or serving.

In some embodiments, the methods result in formulations that comprise Inulin at a dose range of about 0 gm to about 10 gm per daily dose or serving. In some embodiments, the methods result in formulations that comprise Inulin at a dose range of about 0 gm to 10 gm per daily dose or serving.

In some embodiments the methods result in a formulation that is a single serving to be taken once per day by a subject. Thus, in some embodiments a daily dose of active ingredients is provided in a single serving. In some embodiments one daily dose may be divided among two or more servings to be taken in a day, for example among two or more servings of a liquid beverage or two or more tablets. A daily dose of the formulation is preferably taken by or administered to a subject in a 24-hour period. In some embodiments, a daily dose of the formulation is taken every day for 1 week to 12 months. In some embodiments, the formulation is taken for 1, 2, 3 or 4 weeks. In some embodiments, the formulation is taken for 2, 3, 4, 5, 6, 7, 8, 9, 10, 11 or 12 months, or a value within a range defined by any two of the aforementioned values.

In some embodiments, the methods result in a formulation that is to be taken in the morning on an empty stomach. In some embodiments, the methods result in a formulation that is to be taken daily to promote overall health. In some embodiments, the methods result in a formulation that is to be taken to promote a feeling of overall well-being.

In some embodiments, the methods result in a liquid drink formulation such that a single serving of the liquid drink formulation comprises about 4.5 fl. oz. In some embodiments, the methods result in a liquid drink formulation such that a single serving of the liquid drink formulation comprises 4.5 fl. oz. However, the skilled artisan will appreciate that while the size of the serving itself may vary depending on the quantity of the inactive ingredients, such as water, the amount of the active ingredients in each single serving will be within the ranges provided herein. For example, while the single serving size may be ½ fl. oz., 1 fl. oz., 2 fl. oz., 4 fl. oz., 6 fl. oz., 10 fl. oz., 12 fl. oz., 16 fl. oz. or anything smaller or larger, or a value within a range defined by any two of the aforementioned values, the daily dose of active ingredients will be the same in each serving. In some embodiments a single serving is about 2 fl. oz. to about 54 fl. oz. In some embodiments a single serving is 2 fl. oz. to 54 fl. oz. In some embodiments, the formulation is a liquid concentrate comprising of multiple servings to be diluted into a liquid beverage to obtain a single serving size.

In some embodiments, the methods result in a dietary powder supplement comprising a single serving of 7.75 g (1 scoop) (Table 3). In some embodiments, the formulation is a dietary powder supplement comprising a single serving of 1 g-2.5 g. In some embodiments, the formulation is a dietary powder supplement comprising a single serving of 2.5 g-5 g. In some embodiments, the formulation is a dietary powder supplement comprising a single serving of 5 g-7.5 g. In some embodiments, the formulation is a dietary powder supplement comprising a single serving of 7.5 g-10 g. In some embodiments, the formulation is a dietary powder supplement comprising a single serving of 10 g-12.5 g. In some embodiments, the formulation is a dietary powder supplement comprising a single serving of 12.5 g-15 g. In some embodiments, the formulation is a dietary powder supplement comprising a single serving of about 1 g-about 2.5 g. In some embodiments, the formulation is a dietary powder supplement comprising a single serving of about 2.5 g-about 5 g. In some embodiments, the formulation is a dietary powder supplement comprising a single serving of about 5 g-about 7.5 g. In some embodiments, the formulation is a dietary powder supplement comprising a single serving of about 7.5 g-about 10 g. In some embodiments, the formulation is a dietary powder supplement comprising a single serving of about 10 g-about 12.5 g. In some embodiments, the formulation is a dietary powder supplement comprising a single serving of about 12.5 g-about 15 g. In some embodiments, the formulation is a dietary powder supplement comprising a single serving of 1, 2, 3, 4, 5, 6, 7, 8, 9, 10, 11, 12, 13, 14, or 15 g, or a value within a range defined by any two of the aforementioned values. In some embodiments, the formulation is a dietary powder supplement comprising a single serving of about 1, 2, 3, 4, 5, 6, 7, 8, 9, 10, 11, 12, 13, 14, or 15 g, or a value within a range defined by any two of the aforementioned values.

In some embodiments of the methods, the product is cold filled. In some embodiments of the methods, the product is pasteurized. In some embodiments of the methods, the product is pasteurized and/or cold filled.

### Kits of Formulations for Increasing GLP-1 Levels

In some embodiments, a kit is provided which comprises a formulation or combination or subcombination according to any of the embodiments disclosed herein and a dietary plan comprising instructions to use of the formulation in the kit for inhibiting, ameliorating, delaying the onset of, reducing the likelihood of, treating, and/or preventing obesity and diabetes. In some embodiments, the dietary plan comprises taking at least one serving of a formulation provided herein per day, for example reconstituted in the form of a liquid beverage. In some embodiments, the dietary plan can comprise taking at least two servings of a formulation provided herein per day, for example reconstituted in the form of a liquid beverage. In some embodiments, the dietary plan can comprise taking at least one serving of a formulation provided herein per day, for example in the form of a liquid beverage. In some embodiments, at least one serving can be taken in the morning on an empty stomach. In some embodiments, at least one serving can be taken at any time, for example, when increased energy levels are desired (e.g., during a workout, during a marathon, etc.). In some embodiments, at least two servings can be taken at any time. In some embodiments, a maximum of two servings in a 24-hour period can be taken. In some embodiments, at least 1, 2, 3, 4, or 5 servings can be taken at any time. In some embodiments, the servings are effective in inhibiting, ameliorating, delaying the onset of, reducing the likelihood of, treating, and/or preventing obesity and diabetes.

In some embodiments, the kit comprises a formulation in the kit is an edible solid or an edible semi-solid. In some embodiments, the formulation in the kit is a potable liquid. In some embodiments, the formulation in the kit is a powder or pellet that can be reconstituted in a potable liquid (e.g., water). In some embodiments, the formulation in the kit is a tablet or a capsule that can be taken without reconstitution.

### EXAMPLES

The following examples are non-limiting and other variants within the scope of the art also contemplated.

Examples 1-10 provide anecdotal reports of the effect on volunteers of the embodiments of the natural formulations disclosed herein. For all the volunteers, one dosage a day was administered (4 gm of product/formulation), which comprised 1.5 grams cherry extract, 250 mg Vitamin C, 200 mg grape skin extract, and 25 mg Turmeric—otherwise referred in the Examples as Ten Plus.

### Example 1

This study involved a 56 year old female volunteer, whose routine included regular exercises with tennis and Pilates 4-5 times per week. Within one week of starting an embodiment of the formulations disclosed herein (called Ten Plus), she noticed extra stamina and endurance in both tennis performance and Pilates routine. She ran out of her supply for 2-3 days and noticed a dramatic fall in her energy and performance levels. She recovered energy when starting to use Ten Plus again.

### Example 2

This study involved a 57 year old male volunteer, whose regular routine included exercise with Tennis and Pilates with Karen S, and who had a similar experience using Ten Plus for one week. He had a surge in energy and focus while using Ten Plus and had less energy when not using the formulation.

### Example 3

This study involved a 52 year old male volunteer. After one week of using Ten Plus, he noticed extra stamina and endurance in every day activity.

### Example 4

This study involved a 64 year old male volunteer. After one week of using Ten Plus, there were very noticeable changes in energy levels and focus.

### Example 5

This study involved an: 86 year old female volunteer. After three weeks of using Ten Plus, a dramatic increase in energy and stamina was observed. Also, a remarkable drop in C-reactive protein (CRP) and sedimentation rate was observed since using Ten Plus. Prior to taking Ten Plus, her base line level of CRP was 14 mg/L, and her base line sedimentation rate was 55 mm/h. After three weeks of using Ten Plus, the CRP level decreased to 10 mg/L, and sedimentation rate decreased to 20 mm/h.

### Example 6

This study involved a 75 year old male volunteer. After two weeks of using Ten Plus, a strong increase in energy and endurance was observed.

### Example 7

This study involved a 35 year old male volunteer. After one week of using Ten Plus, a dramatic improvement in endurance and focus in work outs was observed.

### Example 8

This study involved a 27 year old male volunteer. After two to three days of using Ten Plus, a very strong increase in energy and endurance in workouts and general stamina.

### Example 9

This study involved a 45 year old female volunteer, who has been lifelong athlete and rigorous workout enthusiast. After three days of using Ten Plus, she noticed a dramatic increase in energy levels.

### Example 10

This study involved a 40 year old female volunteer. After three days of using Ten Plus, she noticed a dramatic increase in endurance and energy and focus.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a formulation having at least one of A, B, and C” would include but not be limited to formulations that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a formulation having at least one of A, B, or C” would include but not be limited to formulations that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. For example, “about 5”, shall include the number 5. Finally, as will be understood by one skilled in the art, a range includes each individual member. Thus, for example, a group having 1-3 values refers to groups having 1, 2, or 3 values. Similarly, a group having 1-5 values refers to groups having 1, 2, 3, 4, or 5 values, and so forth.

It would be understood that the various sizes, materials, configurations and arrangements disclosed herein may be combined and constructed in any way that is feasible to create a hybrid for any particular end use. Accordingly, all suitable modifications and equivalents may be resorted to falling within the scope of the appended claims. Unless defined otherwise, all technical and scientific terms used herein have same meaning as commonly understood by one of ordinary skill in the art to which this application belongs. Also, as used herein and in the appended claims, the singular form “a”, “and”, and “the” include plural referents unless the context clearly dictates otherwise.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. The term “about,” as applied to one or more values disclosed herein, refers to a value that is similar to a stated value. In some embodiments, the term “about” refers to a range of values that fall within 10% or less in either direction (greater than or less than) of the stated value unless otherwise stated or otherwise evident from the context (except where such number would exceed 100% of a possible value). In some embodiments, “about” may mean+/−1-10% of the recited value. For instance, a formulation having about 40 mg of a given ingredient may include 36 mg-44 mg of that ingredient.

It is to be understood that the present invention is not to be limited to the exact description and embodiments as illustrated and described herein. To those of ordinary skill in the art, one or more variations and modifications will be understood to be contemplated from the present disclosure. Accordingly, all expedient modifications readily attainable by one of ordinary skill in the art from the disclosure set forth herein, or by routine experimentation therefrom, are deemed to be within the true spirit and scope of the invention as defined by the appended claims.

What is claimed is:

1. A method for increasing GLP-1 levels in a subject, the method comprising:
administering to the subject a formulation comprising:
an effective amount of at least one extract from a tart cherry;
an effective amount of resveratrol;
an effective amount of L-arginine,
wherein the effective amount of each ingredient is sufficient in combination for increasing GLP-1 levels.

2. The method of claim 1, wherein the effective amount of the at least one extract from a tart cherry ranges from about 250 mg to about 1000 mg.

3. The method of claim 1, wherein the effective amount of the resveratrol ranges from about 100 mg to about 400 mg.

4. The method of claim 1, wherein the effective amount of the L-arginine ranges from about 1.5 g to about 9 g.

5. The method of claim 1, wherein the formulation further comprises an effective amount of at least one antioxidant vitamin.

6. The method of claim 5, wherein the least one antioxidant vitamin is Vitamin C.

7. The method of claim 6, wherein an amount of the Vitamin C ranges from about 25 mg to about 500 mg.

8. The method of claim 1, wherein the formulation further comprises an effective amount of at least one prebiotic.

9. The method of claim 8, wherein the at least one prebiotic is inulin.

10. The method of claim 9, wherein the effective amount of inulin ranges from about 45 mg to about 4500 mg.

11. The method of claim 1, wherein increasing GLP-1 levels inhibits ameliorates, delays an onset of, reduces a likelihood of, treats, and/or prevents diabetes and/or obesity in the subject.

12. A formulation comprising:
an effective amount of at least one extract from a tart cherry;
an effective amount of resveratrol;
an effective amount of L-arginine; and
a pharmaceutically acceptable carrier.

13. The formulation of claim 12, wherein the effective amount of the at least one extract from a tart cherry ranges from about 250 mg to about 1000 mg.

14. The formulation of claim 12, wherein the effective amount of the resveratrol ranges from about 100 mg to about 400 mg.

15. The formulation of claim 12, wherein the effective amount of the L-arginine ranges from about 1.5 g to about 9 g.

16. The formulation of claim 12, wherein the formulation further comprises an effective amount of at least one antioxidant vitamin.

17. The formulation of claim 16, wherein the least one antioxidant vitamin is Vitamin C.

18. The formulation of claim 17, wherein an effective amount of the Vitamin C ranges from about 25 mg to about 500 mg.

19. The formulation of claim 12, wherein the formulation further comprises an effective amount of at least one prebiotic, wherein the at least one prebiotic is inulin.

20. The formulation of claim 19, wherein the effective amount of inulin ranges from about 45 mg to about 4500 mg.
